(19) United States
(12) Patent Application Publication
(10) Pub. No.: US 2020/0054677 A1
(43) Pub. Date: Feb. 20, 2020

(54) T CELLS EXPRESSING CHEMOKINE RECEPTORS FOR TREATING CANCER

(71) Applicant: THE UNIVERSITY OF ADELAIDE, Adelaide, South Australia (AU)

(72) Inventor: Shaun Reuss McColl, Myrtle Bank (AU)

(21) Appl. No.: 16/487,323

(22) PCT Filed: Feb. 21, 2018
(86) PCT No.: PCT/AU2018/050139

§ 371 Date: Aug. 20, 2019

(30) Foreign Application Priority Data
Feb. 21, 2017 (AU) 2017900571

(51) Int. Cl. A61K 35/17; A61P 35/00; C12N 15/86; C07K 14/715; C12N 5/0783

(52) U.S. Cl. CPC A61K 35/17; A61P 35/00; C12N 2740/13043; C07K 14/7158; C12N 5/0636; C12N 15/86

(57) ABSTRACT

The present disclosure relates to methods and products for preventing and/or treating cancer, and in particular to methods, cells and products for preventing and/or treating cancer using adoptive immunotherapies. In certain embodiments, the present disclosure provides a method of treating a subject suffering from, or susceptible to, a cancer associated with chemokine expressing cells, the method comprising exposing the subject to T cells expressing a receptor to the chemokine and thereby treating the subject.

### FIELD

The present disclosure relates to methods and products for preventing and/or treating cancer, and in particular methods, cells and products for preventing and/or treating cancer using adoptive immunotherapies.

### BACKGROUND

The promise of immunotherapies for treating cancers has been recognised for some time. Adoptive immunotherapies, where immune cells are introduced into a subject, appear to be one of the most promising types of immunotherapies. However, despite the clinical benefits of some of these adoptive cell transfer therapies, there are still a variety of limitations to this type of the therapy.

Recently, genetically modified cancer-specific T cells, such as T Cell Receptor transduced T cells and Chimeric Antigen Receptor (CAR) transduced T cells, have been developed to augment adoptive cell transfer immunotherapies against various types of cancers. Although such forms of therapy have exhibited encouraging results in clinical trials, benefits have only been observed in a few types of cancers, such as haematological cancers and melanoma.

In addition, recent results using these type of therapies have only met with limited success in clinical trials for treating solid tumours. In this regard, although anti-tumour effects have been observed in vitro and in animal models, the desired outcomes have not been observed in clinical practice, and there is now intensive research to improve the effectiveness of these type of therapies, particularly for solid tumours.

Accordingly, there is a need for improved or alternative methods and products for treating cancer using adoptive cell transfer immunotherapy.

### SUMMARY

The present disclosure relates to methods and products for preventing and/or treating cancer, and in particular using adoptive immunotherapies.

Certain embodiments of the present disclosure provide a method of treating a subject suffering from, or susceptible to, a cancer associated with chemokine expressing cells, the method comprising exposing the subject to T cells expressing a receptor to the chemokine, and thereby treating the subject.

Certain embodiments of the present disclosure provide a method of preventing and/or treating a cancer associated with chemokine expressing cells in a subject, the method comprising exposing the subject to T cells expressing a receptor to the chemokine, and thereby preventing and/or treating the cancer.

Certain embodiments of the present disclosure provide a method enhancing recruitment of T cells to a tumour, the method comprising expressing in the T cells a receptor to a chemokine expressed by the tumour, and thereby enhancing recruitment of the T cells to the tumour.

Certain embodiments of the present disclosure provide a method of improving targeting of T cells to a tumour, the method comprising expressing in the T cells a receptor to a chemokine expressed by the tumour, and thereby improving targeting of the T cells to the tumour.

Certain embodiments of the present disclosure provide a method of treating a subject suffering from, or susceptible to, a cancer, the method comprising:
determining the chemokine expression of the cancer and/or cells associated with the cancer; and
exposing the subject to T cells expressing a receptor to the chemokine, thereby treating the subject.

Certain embodiments of the present disclosure provide a method of adoptive T cell immunotherapy in a subject suffering from, or susceptible to a cancer associated with chemokine expressing cells, the method comprising exposing the subject to T cells engineered to expressing a receptor to the chemokine and thereby treating the subject by adoptive T cell immunotherapy.

Certain embodiments of the present disclosure provide a method of adoptive T cell immunotherapy in a subject suffering from, or susceptible to a cancer associated with chemokine expressing cells, the method comprising using T cells expressing a receptor to the chemokine for the immunotherapy.

Certain embodiments of the present disclosure provide a therapeutic composition comprising T cells expressing a chemokine receptor.

Certain embodiments of the present disclosure provide isolated T cells engineered to express a chemokine receptor.

Certain embodiments of the present disclosure provide T cells comprising an exogenous nucleic acid expressing a chemokine receptor and/or comprising an exogenous nucleic acid driving expression of an endogenous chemokine receptor gene.

Certain embodiments of the present disclosure provide tumour targeting T cells engineered to express a chemokine receptor.

Certain embodiments of the present disclosure provide use of T cells engineered to express a chemokine receptor for adoptive immunotherapy for treating a cancer.

Certain embodiments of the present disclosure provide a method of producing therapeutic T cells for adoptive immunotherapy for treating a cancer, the method comprising engineering the cells to express a chemokine receptor.

Certain embodiments of the present disclosure provide a chimeric antigen receptor T cell engineered to express a chemokine receptor.

Certain embodiments of the present disclosure provide a combination product comprising;
(i) isolated T cells for adoptive immunotherapy to treat a cancer, wherein the T cells express a chemokine receptor; and
(ii) instructions for administering the T cells to a subject.

Certain embodiments of the present disclosure provide a method of identifying a chemokine receptor for expression in T cells for adoptive immunotherapy for treating a cancer, the method comprising determining the chemokine expression of the cancer and thereby identifying a receptor to the chemokine for expression in the T cells.

Other embodiments are disclosed herein.

### BRIEF DESCRIPTION OF THE FIGURES

Certain embodiments are illustrated by the following figures. It is to be understood that the following description is for the purpose of describing particular embodiments only, and is not intended to be limiting with respect to the description.

FIG. 1 shows γδT17 cells downregulate CCR6 upon activation. (a) Representative flow cytometry of CCR6 and CCR2 expression in skin-draining lymph nodes (sLN) and dermal CD3⁺TCR-γδ+IL-17A-YFP+γδT17 cells from Il17a^{Cre}×Rosa26^{eYFP} mice (n=3). (b) Ex vivo transwell chemotaxis of Il17a^{Cre}×Rosa26^{eYFP} splenic IL-17A+/−γδ T cells to CCL20 and CCL2 (n=3). (c) Representative flow cytometry of CD45⁺γδT17 cells from organs of naïve Il17a^{Cre}×Rosa26^{eYFP} mice (n=3). mLN, mesenteric lymph node; PP, Peyer's patches; siLPL, small intestinal lamina propria lymphocytes. (d) Representative flow cytometry and quantitation of CCR6 and CCR2 expression by γδT17 cells from organs of Il17a^{Cre}×Rosa26^{eYFP} mice either naïve (n=6) or at experimental autoimmune encephalomyelitis (EAE) onset (n=7) or peak (n=5). CNS, central nervous system; iLN, inguinal lymph node; ND, not detected. (e) Representative flow cytometry and quantitation of CCR6 expression by γδT17 cells from wild type (WT) mice given BrdU at d3 post-immunization for EAE, and analysed at d8 (n=4). (f) Representative flow cytometry and frequency of CCR6 and CCR2 expression by γδT17 cells from Il17a^{Cre}×Rosa26^{eYFP} lymphocytes cultured with indicated stimuli for 72 h (n=5). Mean±s.e.m. (a-c) Representative of two experiments. (d,f) Pooled from two experiments. (e) Paired two-tailed Student's t-test, (f) one-way paired ANOVA with Dunnett's multiple comparisons test relative to unstimulated control. *P<0.05, **P<0.01, ***P<0.001, ****P<0.0001.

FIG. 2 shows CCR2 recruits γδT17 cells to inflammatory sites. (a) CD45⁺ CD3⁺ TCRγδ⁺ IL-17A⁺ γδT17 cell numbers in tumour-infiltrating lymphocytes (TIL) following B16 melanoma challenge (n=5/time point). (b) γδT17 cell numbers in TIL d7 post-challenge with B16 melanoma in wild type (WT) (n=12) and Ccr6^{-/-} mice (n=13). (c) γδT17 cell numbers in central nervous system (CNS) at experimental autoimmune encephalomyelitis (EAE) onset in WT (n=7) and Ccr6^{-/-} mice (n=6). (d) γδT17 cell numbers in TIL and inguinal lymph nodes (iLN) d7 post-challenge with B16 melanoma in WT (n=15 (TIL), 9 (iLN)), Ccr2^{-/-} (n=13 (TIL), 10 (iLN)) and Ccr2^{-/-} Ccr6^{-/-} mice (n=9 (TIL), 5 (iLN)). (e) ELISA for CCL2 in tumour supernatant from WT mice challenged with B16 melanoma (n=5/time point). (f) γδT17 cell numbers in CNS and iLN at EAE onset in WT (n=14), Ccr2^{-/-} (n=13) and Ccr2^{-/-} Ccr6^{-/-} mice (n=12). (g) γδT17 cell numbers in CNS at peak disease in WT (n=6), Ccr2^{-/-} (n=5) and Ccr2^{-/-} Ccr6^{-/-} mice (n=6). (h) ELISA for CCL2 in CNS of WT mice with EAE (n=4/time point). (i) Ly5.1 mice (n=4) at d5 post-challenge with B16 melanoma were transferred i.v. with in vitro-expanded γδT17 cells from Ccr2^{-/-} (CD45.2⁺) and F₁ (CD45.1⁺CD45.2⁺) mice. Ccr2^{-/-}:F1 total, Vγ4 and Vγ6 γδT17 cell ratios in spleen and tumours were normalized to input ratio. Vγ4 and Vγ6 gdT17 cells were determined by CD3^{bright} gating. Representative flow cytometry of CD45.2⁺ γδT17 cells at d7 or input. (j) Ly5.1 mice (n=7) at EAE onset were transferred with F₁ and Ccr2^{-/-} γδT17 cells as in (i). Twenty-four hours later, ratios of Ccr2^{-/-}:F1 γδT17 cells in spleen, blood and CNS were normalized to input. Representative flow cytometry of CD45.2⁺ γδT17 cells 24 h later or input. Mean±s.e.m. (a,c,e,i) Representative of two experiments. (b,d,f,j) Pooled from two experiments. (b,c) Unpaired two-tailed Student's t-test, (d,f-g,j) one-way ANOVA with Bonferroni's multiple comparisons test (paired in j)), (i) paired two-tailed Student's t-test. *P<0.05, **P<0.01, ***P<0.001, ****P<0.0001.

FIG. 3 shows CCR2 drives protective γδT17 cell responses. (a) Colony-forming units (c.f.u.) and (b) CD45⁺CD11b⁺Ly6G⁺ neutrophils recovered from nasal wash (NW) of wild type (WT) (n=9) and Tcrd^{-/-} mice (n=10) 72 h post-infection with S. pneumoniae. (c) γδT17 cell numbers in cervical lymph node (cLN) and nasal-associated lymphoid tissue (NALT) and (d) ELISA for CCL2 in digested nasal passage (NP) supernatant in unimmunized mice (n=7) and at 72 h post-S. pneumoniae infection (n=13). (e) Ly5.1 mice (n=5) 24 h post-S. pneumoniae infection were transferred i.v. with expanded γδT17 cells from Ccr2^{-/-} (CD45.2 [symbol] ) and F₁ (CD45.1⁺CD45.2⁺) mice. The Ccr2^{-/-}:F1 γδT17 cell ratio in spleen and NP was normalized to input ratio. (f) Twenty-four hours prior to S. pneumoniae infection, Tcrd^{-/-} hosts received PBS (n=8) or expanded and purified γδT17 cells from WT (n=9) or Ccr2^{-/-} (n=7) mice. c.f.u. recovered from NW 72 h post-infection. Mean±s.e.m. (a-d) Pooled from two experiments. (a,b) Mann-Whitney test, (c,d) unpaired two-tailed Student's t-test, (e) paired two-tailed Student's t-test, (f) Kruskal-Wallis test with Dunn's multiple comparisons test. *P<0.05, **P<0.01.

FIG. 4 shows CCR6 regulates homeostatic γδT17 cell recruitment to dermis. (a) Representative flow cytometry and quantitation of CD45⁺CD3^{lo}TCR-γδ^{lo} (γδT^{lo}) cells from ear skin dermis of naïve wild type (WT) (n=13), Ccr6^{-/-} (n=11), Ccr2^{-/-} (n=10) and Ccr2^{-/-}Ccr6^{-/-} mice (n=5). (b) Representative flow cytometry of Vγ4 expression by dermal γδT^{lo} cells and quantitation of Vγ4⁺ and Vγ4 γδT^{lo} cells in dermis of WT and Ccr6^{-/-} mice (n=7/group). (c) WT or Ccr6^{-/-} lymphocytes were transferred i.v. into naïve Ly5.1 mice (n=4/group). After 36 h, number of CD45.2⁺ γδTlo/γδT17 cells recovered was expressed as % of number transferred. Representative flow cytometry of dermal CD45.2⁺ cells and quantitation of γδT17 cell recovery and Vγ4⁺:Vγ4⁻ ratio (normalized to input). (d) Ccl20 mRNA from whole tissues or sorted CD45⁻ epidermal keratinoctyes (Sca-1⁺Ep-CAM^{lo} interfollicular epidermis (IE), Sca-1^{lo}/⁺CAM⁺ infundibulum and isthmus (IF & IS), Sca-1^{lo}Ep-CAM^{lo} double negative (DN)) or CD45⁻ dermal populations (CD31⁻CD90⁺CD140α⁺ fibroblast, gp38⁺CD31⁺ lymphatic endothelial cells (LEC), gp38^{lo}CD31⁺ blood endothelial cells (BEC), CD31⁻ CD90⁻CD140α⁻ double negative (DN)) from naïve WT mice (pooled from 5 mice/experiment). ND, not detected. Mean±s.e.m. (a,d) Pooled from three experiments, (c) representative of two similar experiments. (a) One-way ANOVA with Bonferroni's multiple comparisons test, (b,c) unpaired two-tailed Student's t-test. *P<0.05, **P<0.01, ***P<0.001, ****P<0.0001.

FIG. 5 shows IRF4 and BATF promote CCR6 downregulation in γδT17 cells. (a) Ccr6 and (b) transcription factor mRNA in sorted γδT17 cells from Il17a^{Cre}×Rosa26^{eYFP} lymphocytes ex vivo or cultured with IL-23/IL-1β for indicated times (pooled from 5 to 7 mice). ND, not detected. (c,d) Expanded γδT17 cells (n=3) were transduced with empty pMIG or pMIG-Rorc retrovirus. (c) Representative flow cytometry of RORγt expression in GFP^{hi} γδT17 cells (gated as in d), relative to isotype (grey) and geometric mean fluorescence intensity (gMFI) relative to GFP fluorescence intensity (FI). (d) Representative flow cytometry of CCR6 expression and quantitation in GFP^{hi} γδT17 cells. (e) Splenocytes from Ly5.1 and either wild type (WT), Irf4^{-/-} or Batf^{-/-} mice were 670 dye-labelled, mixed 50:50 and stimulated with IL-23/IL-1β for 72 h. Representative flow cytometry and quantitation of CCR6 expression and proliferation in CD45.1⁺ or CD45.2⁺ γδT17 cells (n=3/group). (f) Representative flow cytometry and quantitation of CCR6 expression by 670 dye-labelled γδT17 cells from WT splenocytes cultured with IL-23/IL-1β for 72 h with/without mitomycin C pre-treatment (n=3). (a,b) Mean±s.d., (c-f) Mean±s.e.m. (a-f) Representative of two similar experiments. (d,e) Paired two-tailed Student's t-test, (f) one-way paired ANOVA with Bonferroni's multiple comparisons test. *P<0.05, **P<0.01, ***P<0.001.

FIG. 6 shows CCR6 downregulation by γδT17 cells enhances migration to inflamed tissue. (a) Resting lymphocytes from wild type (WT) (n=3) or Ccr6^{-/-} (n=4) mice, or WT lymphocytes stimulated with IL-23/IL-1β for 72 h (n=4) were transferred i.v. into separate naïve Ly5.1 hosts. After 36 h, number of CD45.2⁺ γδT^{lo}/γδT17 cells recovered was expressed as % of number transferred. sLN, skin-draining lymph node. (b) Representative flow cytometry for CCR6 expression by GFP⁺ in vitro-expanded γδT17 cells transduced with empty pMIG or pMIG-Ccr6, relative to isotype (grey) (n=3). (c) Chemotaxis of GFP⁺ γδT17 cells transduced as in (b) to CCL20 (n=3). (d) In vitro-expanded γδT17 cells from F₁ (CD45.1⁺CD45.2⁺) or WT (CD45.2⁺) mice were transduced with empty pMIG or pMIG-Ccr6, respectively. Equal numbers of mixed GFP⁺ cells were transferred i.v. into Ly5.1 mice challenged with B16 melanoma 5 days prior and analysed at d7 (n=5). Representative flow cytometry and ratio of recovered F₁ to WT γδT17 cells within transduced (GFP⁺) and untransduced (GFP⁻) populations. Recovered values were normalized to input values. TIL, tumour-infiltrating lymphocytes. (e,f) In vitro-expanded γδT17 cells from WT or F₁ mice were transduced with empty pMIG or pMIG-Ccr6, respectively. Equal numbers of mixed GFP⁺ cells were transferred i.v. into Ly5.1 mice either (e) 24 h post-infection with S. pneumoniae (n=4) or (f) at experimental autoimmune encephalomyelitis (EAE) onset (n=3) and organs were analysed 48 h later. Ratio of recovered WT to F₁ γδT17 cells within transduced (GFP⁺) and untransduced (GFP⁻) populations, normalized to input values. CNS, central nervous system; NP, nasal passage. Mean±s.e.m. (a) Representative of three similar experiments, (b,d) representative of two experiments. (a) One-way ANOVA with Dunnett's multiple comparisons test relative to resting WT γδT17 cells, (d-f) paired two-tailed Student's t-test. *P<0.05, **P<0.01, ***P<0.001, ****P<0.0001.

### DETAILED DESCRIPTION

The present disclosure relates to methods, cells and products for preventing and/or treating cancer, and in particular using adoptive immunotherapies.

The present disclosure is based, at least in part, upon the recognition that causing expression of selected chemokine receptors in T cells will result in improved trafficking/recruitment of the T cells to tumours expressing those chemokines. It is anticipated that this will lead to improved therapeutic outcomes for patients, particular those suffering from metastasis of solid tumours.

Certain embodiments of the present disclosure provide a method of treating a subject suffering from, or susceptible to, a cancer.

Certain embodiments of the present disclosure provide a method of treating a subject suffering from, or susceptible to, a cancer associated with chemokine expressing cells, the method comprising exposing the subject to T cells expressing a receptor to the chemokine, and thereby treating the subject.

The term “a cancer associated with chemokine expressing cells” as used herein refers to a cancer that comprises cells expressing a chemokine and/or a cancer that is associated with, or affected by, other non-cancerous cells expressing a chemokine which support, affect or invade the cancer. Examples include a cancer comprising chemokine expressing cancerous cells, a cancer where the tumour stroma expresses a chemokine, or a cancer infiltrated with haemopoietic cells expressing a chemokine.

It will also be appreciated that the term “cancer” as used herein includes, for example, a primary cancer, a secondary cancer, a solid cancer, a non-solid cancer, a tumour, and one or more cells (cancerous and/or pre-cancerous) in a tumour, circulating, in lymph and/or at one or more sites in a subject.

In a similar fashion, the term “cells associated with a cancer” as used herein refers to cells in a cancer or tumour, or cells which support, affect or invade a cancer or tumour, and includes cancerous cells, cells in the tumour stroma, or cells infiltrating a cancer.

In certain embodiments, the cancer comprises a cancer having cells expressing a chemokine. In certain embodiments, the cancer comprises a cancer having cancerous cells expressing a chemokine. In certain embodiments, the cancer comprises a cancer where the tumour stroma expresses a chemokine. In certain embodiments, the cancer is a cancer infiltrated with haemopoietic cells expressing a chemokine.

In certain embodiments, the cancer is a primary cancer.

Examples of cancers include melanoma, breast cancer, ovarian cancer, prostate cancer, lung cancer, gastric carcinoma, rhabdomyosarcoma, renal cell carcinoma, glioma, neuroblastoma, squamous cell cancer, head and neck cancer, oesophageal cancer, stomach cancer, bladder cancer, pancreatic cancer, colorectal cancer, renal cancer, osteosarcoma, non-small cell lung cancer, a mesothelioma, a lymphoma, and multiple myeloma. Other cancers are contemplated. In certain embodiments, the cancer excludes one of the aforementioned cancers.

In certain embodiments, the cancer is an ovarian cancer, a breast cancer, a melanoma, a pancreatic cancer or a glioma.

In certain embodiments, the cancer is a metastatic cancer.

Examples of metastatic cancers include melanoma, breast cancer, ovarian cancer, prostate cancer, lung cancer, gastric carcinoma, rhabdomyosarcoma, renal cell carcinoma, glioma, neuroblastoma, squamous cell cancer, head and neck cancer, oesophageal cancer, stomach cancer, bladder cancer, pancreatic cancer, colorectal cancer, renal cancer, osteosarcoma, non-small cell lung cancer, a mesothelioma, a lymphoma and multiple myeloma. Other metastatic cancers are contemplated. In certain embodiments, the metastatic cancer excludes one of the aforementioned metastatic cancers.

In certain embodiments, the metastatic cancer is a metastatic ovarian cancer, a metastatic breast cancer, a metastatic melanoma, a metastatic pancreatic cancer or a metastatic glioma.

In certain embodiments, the cancer is a solid tumour cancer.

In certain embodiments, the cancer is a primary cancer and/or a metastatic cancer.

In certain embodiments, the subject is a human subject. For example, the subject may be a human patient suffering from a metastatic cancer, as described herein.

In certain embodiments, the subject is a mammalian subject, a livestock animal (such as a horse, a cow, a sheep, a goat, a pig), a domestic animal (such as a dog or a cat) and other types of animals such as monkeys, rabbits, mice and laboratory animals. Veterinary applications of the present disclosure are contemplated.

In certain embodiments, the subject is a human subject suffering from a cancer as described herein.

In certain embodiments, the subject is susceptible to a cancer. In certain embodiments, the subject is susceptible to a cancer as described herein.

In certain embodiments, the subject has an increased risk or likelihood of suffering from a cancer. In certain embodiments, the subject has an increased risk or likelihood of suffering from a cancer as described herein. In certain embodiments, the subject has an increased risk or likelihood of suffering from a primary cancer. In certain embodiments, the subject has an increased risk or likelihood of suffering from a metastatic cancer.

The term “T cells” as used herein refers to one or more T cells. It will be appreciated that the term includes a single T cell, multiple T cells, a population of T cells, including a population of substantially identical T cells, a population of T cells for which some of the cells are substantially identical and some are different, or a population of cells in which some cells are T cells. For example, the T cells may be a single T cell expressing one or more chemokine receptors, multiple T cells for which all the T cells have the same profile of chemokine receptors, multiple T cells for which some of the cells have one profile of chemokine receptors and other cells have a different profile of chemokine receptors, a population of T cells for which substantially the T cells have the same profile of chemokine receptors, a population of T cells for which some of the T cells have the same profile of chemokine receptors and other cells are different, or a population of T cells for which some of the cells in the population have one profile of chemokine receptors and other cells (but not necessarily all other cells) have a different profile of chemokine receptors.

In certain embodiments, the T cells comprise CD8⁺ cells, CD4⁺ cells, chimeric antigen receptor T cells (CAR T cells), Natural killer T (NKT) cells, or NK cells. In certain embodiments, the T cells comprise one or more of any of the aforementioned cells or any combination thereof. Other types of T cells are contemplated. Methods for identifying different types of T cells are known in the art.

In certain embodiments the T cells comprise CD8⁺ cells or CD4⁺ cells.

In certain embodiments, the T cells are substantially CD8⁺ cells. In certain embodiments, the T cells are substantially CD4⁺ cells. In certain embodiments, the T cells are substantially CAR T cells.

In certain embodiments, the T cells comprise CD8⁺ CAR T cells. In certain embodiments, the T cells comprise CD4⁺ CAR T cells.

Methods for obtaining different types of T cells are known in the art. For example, CD4⁺ and CD8⁺ T cells may be isolated using a commercially available kit, such as is available from Milentyl Biotec.

CAR T cells may be produced by a method known in the art, for example as described in for example as described in Pule et al. (2008) Nat. Med 14(11): 1264-1270.

In certain embodiments, the T cells are produced from stems cell by a reprogramming technology, such as induced pluripotent stem cells (for example as described in Themeli et al. (2013) Nat. Biotechnol. 31(10): 928-933).

In certain embodiments, the T cells comprise autologous T cells. Methods for obtaining autologous T cells are known in the art.

In certain embodiments, the T cells comprise allogeneic T cells. In certain embodiments, the T cells comprise heterologous T cells. Methods for obtaining allogeneic or heterologous cells are known in the art.

In certain embodiments, the T cells comprise in vitro cells. In certain embodiments, the T cells comprise in vitro cultured cells. Methods for culturing T cells in vitro are known in the art.

In certain embodiments, the T cells comprise cells expanded or proliferated in vitro. Methods for expanding/proliferating T cells are known in the art.

In certain embodiments, the T cells comprise purified and/or enriched cells. Methods for purifying/enriching cells are known in the art, for example by using flow cytometry using an appropriate marker.

In certain embodiments, the T cells comprise ex vivo cells.

In certain embodiments, the T cell comprise in vivo cells.

In certain embodiments, the T cells comprise cells enriched or purified using the expressed chemokine receptor. For example, antibodies to chemokine receptors are known in the art and may be used to enrich or purify cells expressing the particular chemokine receptor, such as by use of flow cytometry.

In certain embodiments, the T cells comprise cells that are enriched or purified using another marker in the T cell associated with expression of the chemokine receptor, such as a co-transfected fluorescent marker.

In certain embodiments, the T cells comprise T cells enriched by a method comprising flow cytometry. For example, cells may be enriched by use of fluorescent activated cell sorting using an antibody to a cell surface expressed marker(s), or using a fluorescent marker expressed in the cell to sort the cells. Methods for cell sorting are known in the art, for example as described in “Current Protocols in Cytometry” ed. J. Paul Robinson et al. (1999-2016), John Wiley & Sons. Other methods for purifying/enriching cells are known in the art, such as those based on affinity or physical/biological parameters.

The term “chemokine receptor” as used herein refers to a receptor that interacts with a chemokine, and also includes functional parts or fragments of a receptor. The term also includes chemokine receptors from different species from the species in which the cancer originates, naturally occurring receptors, synthetic variants, a hybrid receptor, homologues, orthologues and paralogues of receptors, variants of receptors (including substitutions, insertions, deletions, fusions, parts of a receptor, non-naturally occurring amino acid variants, and combinations thereof), and modified forms of receptors, such as post-translation modified forms.

In certain embodiments, the chemokine expressing cancer is a type of cancer recognised to be associated with the expression of a specific chemokine(s). For example, in a subject diagnosed with a specific type cancer, the type of cancer may be known to be associated with the expression of a certain chemokine, and accordingly T cells expressing a receptor to that chemokine may be administered to the subject. Examples of chemokines known to be associated with certain types of cancers are provided in Table 2 herein.

In certain embodiments, the cancer is a type of cancer for which the chemokine expression is unknown and which investigation to determine what profile of chemokine(s) are expressed by the cells associated with the cancer is required. For example, in a subject suffering from a cancer, a biopsy of the cancer may be taken from the subject and the chemokine expression of the cancer determined by a method known in the art. Methods for determining the profile of chemokine expression typically involve use of an immunodetection method and/or the use of RNA expression analysis. Once the chemokine expression of the cancer has been identified, T cells expressing the receptor to the chemokine may be administered to the subject. Methods for determining chemokine expression in cancers are known in the art.

Examples of chemokine receptors and their associated ligands are provided in Table 1.

TABLE 1

| CC Chemokine | |
|---|---|
| Receptor | Chemokine ligand |
| CCR1 | CCL3, CCL3L1, CCL5, CCL8, |
| | CCL14, CCL15, CCL16 CCL23 |
| CCR2 | CCL2, CCL7, CCL8, CCL11, |
| | CCL16 |
| CCR3 | CCL3L1, CCL5, CCL7, CCL11, |
| | CCL13, CCL14, CCL15, CCL24, |
| | CCL28 |
| CCR4 | CCL17, CCL22 |
| CCR5 | CCL3, CCL3L1, CCL4, CCL4L1, |
| | CCL5, CCL8, CCL11, CCL16 |
| CCR6 | CCL20 |
| CCR7 | CCL19, CCL21 |
| CCR8 | CCL1, CCL18 |
| CCR9 | CCL25 |
| CCR10 | CCL27, CCL28 |
| | Chemokine Ligand |
| CXC Chemokine | |
| Receptor | |
| CXCR1 | CXCL6, CXCL7, CXCL8 |
| CXCR2 | CXCL1, CXCL2, CXCL3, CXCL5, |
| | CXCL6, CXCL7, CXCL8 |
| CXCR3 | CXCL4, CXCL9, CXCL10, |
| | CXCL11, CXCL13 |
| CXCR4 | CXCL12 |
| CXCR5 | CXCL13 |
| CXCR6 | CXCL16 |
| XC Chemokine | |
| Receptor | |
| XCR1 | XCL1, XCL2 |
| CX₃C Chemokine | |
| Receptor | |
| CX3CR1 | CX3CL1, CCL26 |

As described herein, T cells expressing a chemokine receptor may be used as a tailored immunotherapy for specific cancers.

In certain embodiments, the T cell expresses a single chemokine receptor. In certain embodiments, the T cell expresses two chemokine receptors. In certain embodiments, the T cell expresses two or more chemokine receptors. In certain embodiments, the T cell expresses multiple chemokine receptors.

In certain embodiments, the chemokine receptor is one or more of the chemokine receptors listed in Table 1. In certain embodiments, the chemokine receptor is two or more of the chemokine receptors listed in Table 1. Combinations of any two or more of the chemokine receptors listed in Table 1 is contemplated.

In certain embodiments, the receptor to the chemokine is one or more of CCR2, CXCR3, CCR6, CCR9, CCR10, CXCR4, CXCR6, CXCR5, XCR1 and CCR5. Combinations of any two or more of the aforesaid chemokine receptors are contemplated.

Examples of combinations of chemokine receptors include CCR2 and CXCR3, CCR2 and CCR6, CCR2 and CCR9, CCR2 and CCR10, CCR2 and CXCR4, CCR2 and CXCR6, CCR2 and CXCR5, CCR2 and XCR1, CCR2 and CCR5, CXCR3 and CCR6, CXCR3 and CCR9, CXCR3 and CCR10, CXCR3 and CXCR4, CXCR3 and CXCR6, CXCR3 and CXCR5, CXCR3 and XCR1, CXCR3 and CCR5, CCR6 and CCR9, CCR6 and CCR10, CCR6 and CXCR4, CCR6 and CXCR6, CCR6 and CXCR5, CCR6 and XCR1, CCR6 and CCR5, CCR9 and CCR10, CCR9 and CXCR4, CCR9 and CXCR6, CCR9 and CXCR5, CCR9 and XCR1, CCR9 and CCR5, CCR10 and CXCR4, CCR10 and CXCR6, CCR10 and CXCR5, CCR10 and XCR1, CCR10 and CCR5, CXCR4 and CXCR6, CXCR4 and CXCR5, CXCR4 and XCR1, CXCR4 and CCR5, CXCR6 and CXCR5, CXCR6 and XCR1, CXCR6 and CCR5, CXCR5 and XCR1, CXCR5 and CCR5, and XCR1 and CCR5.

In certain embodiments, a single chemokine receptor is expressed in a T cell. In certain embodiments, two or more chemokine receptors are expressed in a T cell.

In certain embodiments, the chemokine expressed by cells associated with the cancer comprises one or more of CCL2, CCL7, CCL8, CCL11, and CCL16 and the chemokine receptor expressed in the T cells comprises CCR2.

In certain embodiments, the chemokine expressed by cells associated with the cancer comprises one or more of CXCL4, CXCL9, CXCL10, CXCL11, and CXCL13 and the chemokine receptor expressed in the T cells comprises CXCR3.

In certain embodiments, the chemokine expressed by cells associated with the cancer comprises CCL20 and the chemokine receptor expressed in the T cells comprises CCR6.

In certain embodiments, the chemokine expressed by cells associated with the cancer comprises CCL25 and the chemokine receptor expressed in the T cells comprises CCR9.

In certain embodiments, the chemokine expressed by cells associated with the cancer comprises CCL27 and/or CCL28 and the chemokine receptor expressed in the T cells comprises CCR10.

In certain embodiments, the chemokine expressed by cells associated with the cancer comprises one or more of CCL3, CCL3L1, CCL4, CCL4L1, CCL5, CCL8, CCL11, and CCL16 and the chemokine receptor expressed in the T cells comprises CCR5.

In certain embodiments, the chemokine expressed by cells associated with the cancer comprises CXCL16 and the chemokine receptor expressed in the T cells comprises CXCR6.

In certain embodiments, the chemokine expressed by cells associated with the cancer comprises CXCL12 and the chemokine receptor expressed in the T cells comprises CXCR4.

In certain embodiments, the chemokine expressed by cells associated with the cancer comprises CXCL13 and the chemokine receptor expressed in the T cells comprises CXCR5.

In certain embodiments, the chemokine expressed by cells associated with the cancer comprises XCL1 and/or XCL2 and the chemokine receptor expressed in the T cells comprises XCR1.

Other combinations of chemokines expressed in a cancer (and cells associated with the cancer) and chemokine receptors are contemplated.

In certain embodiments, the chemokine receptor is not CXCR6. In certain embodiments, the chemokine receptor is selected from a chemokine receptor excluding CXCR6.

In certain embodiments, the chemokine receptor is not CCR2. In certain embodiments, the chemokine receptor is selected from a chemokine receptor excluding CCR2.

In certain embodiments, the chemokine receptor is not CX3CR1. In certain embodiments, the chemokine receptor is selected from a chemokine receptor excluding CX3CR1.

In certain embodiments, the chemokine receptor is not CXCR4. In certain embodiments, the chemokine receptor is selected from a chemokine receptor excluding CXCR4.

In certain embodiments, the chemokine receptor is not CCR4. In certain embodiments, the chemokine receptor is selected from a chemokine receptor excluding CCR4.

Methods for expressing a chemokine receptor in T cells, such as CAR-T cells, are known in the art. For example, a gene for a specific chemokine receptor (or part thereof), or an appropriate cDNA, from a suitable source may be obtained by a method known in the art or from a commercially available source, for expression in T cells and cloned into a suitable expression vector.

For example, cloned human chemokine receptors (such as CCR2CXCR3, CCR6, CCR9, CCR10, CXCR4, CXCR6, CXCR5, XCR1 and CCR5) may be obtained commercially and cloned into a suitable vector for expression in target cells. Typically, the vector contains a suitable promoter and/or other elements for expression of a cloned product in the desired target cells. For example, a nucleic acid encoding one or more of CCR2, CXCR3, CCR6, CCR9, CCR10, CXCR4, CXCR6, CXCR5, XCR1 or CCR5 may be cloned into a suitable retroviral vector for expression in the T cells. Examples of promoters for expression in T cells include viral promoters (such as cytomegalovirus [CMV] and murine stem cell virus [MSCV]), cellular promoters (such as phosphoglycerate kinase [PGK]), and composite promoters (such as a composite promoter sequence comprised of the CMV enhancer and portions of the chicken beta-actin promoter and the rabbit beta-globin gene, and SV40/CD43). In circumstances where two or more chemokine receptors are cloned into a single vector, additional genes may be expressed utilising for example an internal ribosome entry site. Similar constructs may be utilised for other chemokine receptors, such as those described herein.

Genomic and mRNA sequences of chemokine receptors are publicly available. Examples are provided herein.

The HUGO Gene Nomenclature Committee accession number for the human CCR2 gene is HGNC:1603. The Genbank accession number for the human mRNA (isoform A) is NM_01123041.2 and the human protein is NP_001116513.2. The Genbank accession number for isoform B for the mRNA is NM_011233396.1 and Genbank accession number for the protein is NP_001116868. The CCR2 gene, mRNAs and encoded proteins in other species may be identified by a method known in the art, such as the BLAST suite of algorithms, for example as described in Altschul, S et al. (1990). Journal of Molecular Biology 215 (3): 403-410. CCR2 constructs for use to express the protein in T cells may be produced by a method known in the art or be commercially obtained, such as from InvivoGen (human CCR2 Cat. #puno1-hccr2; mouse CCR2 Cat #puno1-mccr2,).

The HUGO Gene Nomenclature Committee accession number for human CXCR3 is HGNC 4540. The Genbank accession number for the human mRNA (isoform 1) is NM_001504.1. The CXCR3 gene, mRNAs and encoded proteins in other species may be identified by a method known in the art. CXCR3 constructs for use to express the protein in T cells may be produced by a method known in the art or be commercially obtained, such as from InvivoGen (Human CXCR3 Cat #puno1-hcxcr3; mouse CXCR3 Cat #puno1-mcxcr3). The CXCR3 gene, mRNAs and encoded proteins in other species may be identified by a method known in the art, such as the BLAST suite of algorithms, for example as described in Altschul, S et al. (1990). Journal of Molecular Biology 215 (3): 403-410.

The HUGO Gene Nomenclature Committee accession number for human CCR9 is HGNC 6110. The Genbank accession number for the human mRNA is NM_031200.2. The CCR9 gene, mRNAs and encoded proteins in other species may be identified by a method known in the art. CCR9 constructs for use to express the protein in T cells may be produced by a method known in the art or be commercially obtained, such as from InvivoGen (human CCR9 Cat #puno1-hccr9; mouse CCR9 Cat #puno1-mccr9). The CCR9 gene, mRNAs and encoded proteins in other species may be identified by a method known in the art, such as the BLAST suite of algorithms, for example as described in Altschul, S et al. (1990). Journal of Molecular Biology 215 (3): 403-410.

The HUGO Gene Nomenclature Committee accession number for human CCR10 is HGNC 4474. The Genbank accession number for the human mRNA is NM_016602.2). The CCR10 gene, mRNAs and encoded proteins in other species may be identified by a method known in the art. CCR10 constructs for use to express the protein in T cells may be produced by a method known in the art or be commercially obtained, such as from InvivoGen (human CCR10 Cat #puno1-hccr10). The CCR10 gene, mRNAs and encoded proteins in other species may be identified by a method known in the art, such as the BLAST suite of algorithms, for example as described in Altschul, S et al. (1990). Journal of Molecular Biology 215 (3): 403-410.

The HUGO Gene Nomenclature Committee accession number for human CCR5 is HGNC 1606. The Genbank accession number for the human mRNA is NM_000579.3). The CCR5 gene, mRNAs and encoded proteins in other species may be identified by a method known in the art. CCR5 constructs for use to express the protein in T cells may be produced by a method known in the art or be commercially obtained, such as from InvivoGen (human CCR5 Cat #puno1-hccr5; mouse CCR5, Cat #puno1-mccr5). The CCR5 gene, mRNAs and encoded proteins in other species may be identified by a method known in the art, such as the BLAST suite of algorithms, for example as described in Altschul, S et al. (1990). Journal of Molecular Biology 215 (3): 403-410.

The HUGO Gene Nomenclature Committee accession number for human CCR6 is HGNC 1607. The Genbank accession number for human mRNA is isoform 1 is NM_000579.3. The CCR6 gene, mRNAs and encoded proteins in other species may be identified by a method known in the art. CCR6 constructs for use to express the protein in T cells may be produced by a method known in the art or be commercially obtained, such as from InvivoGen (Human CCR6 Cat #puno1-hccr6; mouse CCR6 Cat #puno1-mccr6). The CCR6 gene, mRNAs and encoded proteins in other species may be identified by a method known in the art, such as the BLAST suite of algorithms, for example as described in Altschul, S et al. (1990). Journal of Molecular Biology 215 (3): 403-410.

The HUGO Gene Nomenclature Committee accession number for human CXCR4 is HGNC 2561. The Genbank accession number for human mRNA is isoform 1 is NM_001008540.1. The CXCR4 gene, mRNAs and encoded proteins in other species may be identified by a method known in the art. CCR6 constructs for use to express the protein in T cells may be produced by a method known in the art or be commercially obtained, such as from InvivoGen (Human CXCR4 Cat #puno1-hcxcr4; mouse CXCR4 Cat #puno1-mcxcr4). The CXCR4 gene, mRNAs and encoded proteins in other species may be identified by a method known in the art, such as the BLAST suite of algorithms, for example as described in Altschul, S et al. (1990). Journal of Molecular Biology 215 (3): 403-410.

The HUGO Gene Nomenclature Committee accession number for human CXCR6 is HGNC 1647. The Genbank accession number for human mRNA is isoform 1 is NM_00656.1 The CXCR6 gene, mRNAs and encoded proteins in other species may be identified by a method known in the art. CCR6 constructs for use to express the protein in T cells may be produced by a method known in the art or be commercially obtained, such as from InvivoGen (Human CXC6 Cat #puno1-hcxcr6; mouse CXCR6 Cat #puno1-mcxcr6). The CXCR6 gene, mRNAs and encoded proteins in other species may be identified by a method known in the art, such as the BLAST suite of algorithms, for example as described in Altschul, S et al. (1990). Journal of Molecular Biology 215 (3): 403-410.

The HUGO Gene Nomenclature Committee accession number for human CXCR5 is HGNC 1060. The Genbank accession number for human mRNA, transcript variant 1, is NM_001716. The CXCR5 gene, mRNAs and encoded proteins in other species may be identified by a method known in the art. CXCR5 constructs for use to express the protein in T cells may be produced by a method known in the art or be commercially obtained, such as from InvivoGen (Human CXCR5 Cat #puno1-hcxcr5). The CXCR5 gene, mRNAs and encoded proteins in other species may be identified by a method known in the art, such as the BLAST suite of algorithms, for example as described in Altschul, S et al. (1990). Journal of Molecular Biology 215 (3): 403-410.

The HUGO Gene Nomenclature Committee accession number for human XCR1 is HGNC 625. The Genbank accession number for human mRNA, transcript variant 1, is NM_001024644. The XCR1 gene, mRNAs and encoded proteins in other species may be identified by a method known in the art. XCR1 constructs for use to express the protein in T cells may be produced by a method known in the art or be commercially obtained, such as from InvivoGen (Human XCR1 Cat #puno1-hxcr1). The XCR1 gene, mRNAs and encoded proteins in other species may be identified by a method known in the art, such as the BLAST suite of algorithms, for example as described in Altschul, S et al. (1990). Journal of Molecular Biology 215 (3): 403-410.

Examples of suitable expression vectors are known in the art and include retroviral vectors, lentiviral vectors, adenoviral vectors, and AAV vectors. Methods for cloning into vectors are known in the art, for example as described in Green M R and Sambrook J, Molecular Cloning: A Laboratory Manual (4th edition), Cold Spring Harbor Laboratory Press, 2012., and Ausubel et al., Current Protocols in Molecular Biology (2011), John Wiley & Sons, Inc., both of which are herein incorporated by reference.

Vectors encoding a chemokine receptor, (eg one or more of CCR2, CXCR3, CCR6, CCR9, CCR10, CXCR4, CXCR6, CXCR5, XCR1 and CCR5) for expression may be introduced into T cells by a method known in the art, for example by transduction. For example, lentiviral vectors for gene transfer into T cells are described in Verhoeyen et al. (2009) Methods Mol. Biol. 506: 97-114, and Jones et al. (2009) Hum. Gene Ther. 20(6): 630-640.

It will be appreciated that chemokine receptors from the same species or different species may be expressed in a T cell. For example, a human chemokine receptor may be expressed in a human T cell, a non-human chemokine receptor may be expressed in a human T cell, or a human chemokine receptor may be expressed in a non-human T cell. As described herein, the chemokine receptor may be a full length receptor, a modified receptor, a fragment of a receptor, a hybrid receptor, or a variant of a receptor. Other forms of a receptor are contemplated.

In certain embodiments, the T cells comprise a virally transduced nucleic acid encoding a chemokine receptor(s).

In certain embodiments, the T cells comprise a virally transduced nucleic acid encoding a chemokine receptor selected from one or more of CCR2, CXCR3, CCR6, CCR9, CCR10, CXCR4, CXCR6, CXCR5, XCR1 and CCR5.

In certain embodiments, the T cells comprise an exogenous chemokine receptor selected from one or more of CCR2, CXCR3, CCR6, CCR9, CCR10, CXCR4, CXCR6, CXCR5, XCR1 and CCR5.

In certain embodiments, the T cells comprise T cells expressing an exogenous chemokine receptor virally transduced into the cells.

In certain embodiments, the T cells comprise an exogenous nucleic acid expressing a chemokine receptor and/or comprise an exogenous nucleic acid driving expression of an endogenous chemokine receptor gene. Methods for using an exogenous nucleic acid to drive expression of an endogenous gene are known in the art.

In certain embodiments, the T cells comprise an exogenous nucleic acid expressing a chemokine receptor selected from one or more of CCR2, CXCR3, CCR6, CCR9, CCR10, CXCR4, CXCR6, CXCR5, XCR1 and CCR5 and/or comprise an exogenous nucleic acid driving expression of an endogenous chemokine receptor selected from one or more of a CCR2, CXCR3, CCR6, CCR9, CCR10, CXCR4, CXCR6, CXCR5, XCR1 and CCR5 gene.

In certain embodiments, the T cells expressing a chemokine receptor comprise T cells engineered to express the receptor. Methods for engineering T cells are known in the art.

In certain embodiments, the T cells expressing a chemokine receptor selected from one or more of CCR2, CXCR3, CCR6, CCR9, CCR10, CXCR4, CXCR6, CXCR5, XCR1 and CCR5 comprise cells engineered to express one or more of the aforementioned chemokine receptors.

In certain embodiments, the T cells comprise an exogenous chemokine receptor encoding nucleic acid. In certain embodiments, the T cells comprise an exogenous CCR2, CXCR3, CCR6, CCR9, CCR10, CXCR4, CXCR6, CXCR5, XCR1 or CCR5 encoding nucleic acid.

In certain embodiments, the T cells further comprise one or more exogenous markers to assist with enrichment of the cells.

Methods for detecting chemokine receptor expression in cells are known in the art. For example an antibody to a chemokine receptor may be used to detect expression of the receptor in T cells. Antibodies to chemokine receptors are commercially available. Analysis of mRNA expression may also be used. Probes suitable for detecting mRNAs are commercially available or may be produced by a method known in the art.

As will be appreciated, an appropriate level of expression of a chemokine receptor chemokine receptor in the T cells may be selected, for example a level sufficient to promote targeting/trafficking of the T cells to tumours. Methods for determining expression are known in the art and include analysis of cell surface expression.

In certain embodiments, the exposing of the subject to T cells expressing a receptor to a chemokine comprises administering T cells expressing the chemokine to the subject.

In certain embodiments, the exposing of the subject to T cells expressing a receptor to a chemokine comprises causing T cells in vivo to express a chemokine receptor.

Other methods of exposing a subject to T cells are contemplated.

Methods for administering cells to a subject are known in the art, for example as described in “Adoptive Immunotherapy—Methods and Protocols” (2010) Edited by B. Ludewig and M. W. Hoffman, Humana Press.

A suitable regime for exposing/administering the T cells to a subject may be chosen.

For administration of T cells to a subject, the cells may be administered by a suitable route and in a suitable form.

In certain embodiments, the cells are administered intravenously. In certain embodiments, the cells are administered via injection, such as by intravenous injection, or by intravenous infusion. In this case, the administration of the cells may for example utilise a liquid vehicle, such as isotonic saline. Methods for administering cells are known in the art. Other methods of administration are contemplated.

The cells may be administered alone or may be delivered in a mixture with a vehicle, carrier, one or more other therapeutic agents and/or one or more agents that enhance, stabilise or maintain the activity of the cells being delivered. In certain embodiments, an administration vehicle (e.g. an injectable solution) would contain the cells, and optionally excipient(s) and additional agent(s). For example, a suitable administration vehicle may comprise isotonic saline.

The methods described herein may also include combination therapy. In this regard, the subject may be treated or given another agent/drug or treatment modality in conjunction with the cells as described herein. Such combination therapy can be sequential therapy where the subject is treated first with one and then the other, or the two or more treatment modalities are given simultaneously.

The therapeutically effective amount of cells for use in the methods as described herein may be selected, and may vary depending upon the particular cells utilized, the mode of administration, the cancer and severity thereof, as well as the various physical factors related to the subject being treated.

Dosages of the T cells are expected to vary with parameters such as route of administration, and the nature of the cells administered. In certain embodiments, the method comprises administering to the subject a single dose of cells, or repeated doses. Suitable regimes may be selected. In certain embodiments, the cells are administered via injection, such as intravenous injection, direction injection into a target site or by intravenous infusion.

In certain embodiments, the method comprises administering 10⁸ to 10¹² T cells to the subject. In certain embodiments, the method comprises administering 10⁹ to 10¹¹ T cells to the subject.

In certain embodiments, the method comprises administering 10⁸ to 10¹¹, 10⁸ to 10¹⁰, 10⁸ to 10⁹, 10⁹ to 10¹², 10⁹ to 10¹¹, 10⁹ to 10¹⁰, 10¹⁰ to 10¹², 10¹⁰ to 10¹¹, or 10¹¹ to 10¹² T cells to the subject. Other amounts of T cells for administration to the subject are contemplated.

In certain embodiments, the exposing of the subject to the T cells comprises producing suitable T cells in vivo that express the chemokine receptor, or causing suitable T cells in vivo to express the chemokine receptor. For example, infection of the subject with a suitable virus carrying a chemokine receptor gene/cDNA may be used in order to express the chemokine receptor in the target cells, using methods known in the art. Viral vectors and their use in gene therapy are as described, for example, in “Viral Vectors for Gene Therapy, Methods and Protocols” Editors Merten, Otto-Wilhelm, Al-Rubeai, Mohamed (2011) Humana Press. Other methods for gene delivery are contemplated, for example as described in “Non-Viral Gene Therapy” by H. Summers (2015) published by Foster Academics.

In certain embodiments, a method as described herein is used to prevent and/or treat a cancer in a subject.

In certain embodiments, the present disclosure provides a method of preventing and/or treating a cancer associated with chemokine expressing cells in a subject, the method comprising exposing the subject to T cells expressing a receptor to the chemokine, and thereby preventing and/or treating the cancer.

The term “preventing”, and related terms such as “prevention” and “prevent”, as used herein refers to obtaining a desired therapeutic and/or physiologic effect in terms of arresting or suppressing the appearance of one or more symptoms in the subject. Methods for assessing prevention are known in the art.

The term “treatment”, and related terms such as “treating” and “treat”, as used herein refers to obtaining a desired therapeutic and/or physiologic effect in terms of improving the condition of the subject, ameliorating, arresting, suppressing, relieving and/or slowing the progression of one or more symptoms in the subject, a partial or complete stabilization of the subject, a regression of one or more symptoms, or a cure of the subject. Methods for assessing treatment are known in the art.

The term “exposing”, and related terms such as “expose” and “exposure” as used herein refers to bringing one agent into contact with, or the vicinity of, a target, and includes methods of exposure such as administration of an agent to a subject or producing a desired agent in a subject.

Cancers are as described herein. T cells and methods for exposing the cells to subjects are as described herein.

In certain embodiments, a method as described herein is used to enhance/promote recruitment of T cells to a tumour.

In certain embodiments, the present disclosure provides a method enhancing recruitment of T cells to a tumour, the method comprising expressing in the T cells a receptor to a chemokine expressed by the tumour (and/or cells associated with the tumour), and thereby enhancing recruitment of the T cells to the tumour.

In certain embodiments, the tumour is a melanoma tumour, a breast cancer tumour, an ovarian cancer tumour, a prostate cancer tumour, a lung cancer tumour, a gastric carcinoma tumour, a rhabdomyosarcoma tumour, a renal cell carcinoma tumour, a glioma tumour, a neuroblastoma tumour, a squamous cell cancer tumour, ha head and neck cancer tumour, an oesophageal cancer tumour, a stomach cancer tumour, a bladder cancer tumour, a pancreatic cancer tumour, a colorectal cancer tumour, a renal cancer tumour, an osteosarcoma tumour, a non-small cell lung cancer tumour, a mesothelioma tumour and a multiple myeloma tumour. Other types of tumours are contemplated.

T cells, and methods for exposing T cells to a subject, are as described herein.

Methods for assessing the recruitment of T cells to a tumour are known in the art. For example, staining of tumour biopsies to identify T cells that have infiltrated into the tumour are known in the art.

In certain embodiments, the recruitment of cells to the tumour is sufficient to cause regression of the tumour.

In certain embodiments, a method as described herein may be used to improving targeting of T cells to a tumour.

In certain embodiments, the present disclosure provide a method of improving targeting of T cells to a tumour, the method comprising expressing in the T cells a receptor to a chemokine expressed by the tumour (and/or cells associated with the tumour), and thereby improving targeting of the T cells to the tumour.

In certain embodiments, the tumour is a melanoma tumour, a breast cancer tumour, an ovarian cancer tumour, a prostate cancer tumour, a lung cancer tumour, a gastric carcinoma tumour, a rhabdomyosarcoma tumour, a renal cell carcinoma tumour, a glioma tumour, a neuroblastoma tumour, a squamous cell cancer tumour, head and neck cancer tumour, an oesophageal cancer tumour, a stomach cancer tumour, a bladder cancer tumour, a pancreatic cancer tumour, a colorectal cancer tumour, a renal cancer tumour, an osteosarcoma tumour, a non-small cell lung cancer tumour, a mesothelioma tumour and a multiple myeloma tumour. Other tumours are contemplated.

Methods for assessing targeting of T cells to a tumour are known in the art, for example the presence of the T cells in a tumour may be determined.

In certain embodiments, the expression of chemokines in the cancer and/or cells associated with the cancer may be determined. Methods for determining the chemokine expression of cancers/cells are known in the art.

In certain embodiments, the present disclosure provides a method of treating a subject suffering from, or susceptible to, a cancer, the method comprising:
determining the chemokine expression of the cancer and/or cells associated with the cancer; and
exposing the subject to T cells expressing a receptor to the chemokine, thereby treating the subject.

Certain embodiments of the present disclosure provide methods of adoptive T cell immunotherapy using T cells as described herein.

Methods for performing adoptive immunotherapy are known in the art, for example as described in as described in “Adoptive Immunotherapy—Methods and Protocols” (2010) Edited by B. Ludewig and M. W. Hoffman, Humana Press.

In certain embodiments, the present disclosure provides a method of adoptive T cell immunotherapy in a subject suffering from, or susceptible to a chemokine expressing cancer, the method comprising exposing the subject to T cells engineered to express a receptor to the chemokine and thereby treating the subject by adoptive T cell immunotherapy.

In certain embodiments, the present disclosures provides a method of adoptive T cell immunotherapy in a subject suffering from, or susceptible to a chemokine expressing cancer, the method comprising using T cells expressing a receptor to the chemokine for the immunotherapy.

In certain embodiments, T cells as described herein are used in a therapeutic composition or a medicament.

Certain embodiments of the present disclosure provide a therapeutic composition comprising T cells expressing a chemokine receptor.

In certain embodiments, the T cells express a chemokine receptor selected from one or more of CCR2, CXCR3, CCR6, CCR9, CCR10, CXCR4, CXCR6, CXCR5, XCR1 and CCR5, or combination of any one or more of the aforementioned chemokine receptors.

Compositions of T cells are as described herein. T cells expressing a chemokine receptor(s), and methods for producing such cells, are as described herein.

In certain embodiments, the therapeutic composition is used in a method of the present disclosure.

For example, a therapeutic composition may comprise T cells expressing one or more of CCR2, CXCR3, CCR6, CCR9, CCR10, CXCR4, CXCR6, CXCR5, XCR1 and CCR5 in physiological saline. In certain embodiments, the therapeutic composition comprises T cells expressing a combination of different receptors.

In one example, a therapeutic composition may comprise T cells expressing the same chemokine receptors. In another example, the T cells may comprise some T cells expressing one or more specific chemokine receptors and other T cells expressing one or more different receptors.

In certain embodiments, the composition comprises 10⁸ to 10¹¹, 10⁸ to 10¹⁰, 10⁸ to 10⁹, 10⁹ to 10¹², 10⁹ to 10¹¹, 10⁹ to 10¹⁰, 10¹⁰ to 10¹², 10¹⁰ to 10¹¹, or 10¹¹ to 10¹² T cells. Other amounts of T cells are contemplated.

In certain embodiments, the composition is suitable for delivery to the subject, for example by way of intravenous administration.

Methods for formulating suitable compositions are known in the art.

In certain embodiments, the cells are provided with an acceptable carrier suitable for administering the cells to a subject. Carriers may be chosen based on the route of administration as described herein, the type of cells being delivered, the time course of delivery etc. The term “acceptable carrier” refers to a substantially inert carrier. An example of an acceptable carrier is physiological saline. Other physiologically acceptable carriers and their formulations are known in the art.

In certain embodiments, a composition or medicament comprises other therapeutic agents and/or agents that enhance, stabilise or maintain the activity of the cells and/or their active component(s).

In certain embodiments, a cancer as described herein may be prevented and/or treated using a composition or medicament as described herein.

In certain embodiments, the present disclosure provides a method of preventing and/or treating a cancer associated with chemokine expressing cells in a subject, the method comprising exposing the subject to a therapeutic composition as described herein.

Certain embodiments of the present disclosure provide isolated T cells engineered to express a chemokine receptor.

The term “isolated” or the related terms such as “isolate” or “isolating” as used herein refer to a process whereby a species, such as a cell, has been separated (partially or completely) from its natural or original environment.

For example, an isolated cell may be in a substantially purified state, or be a cell in a population of other cells. In certain embodiments, the isolated T cells engineered to express a chemokine receptor comprise at least 10%, 20%, 30%, 40%, 50%, 60%, 70%, 80%, 90%, or 95% of cells in a total cell population.

Methods for isolating T cells are known in the art. In certain embodiments, the method of isolating cells comprises one or more of flow cytometry, cell sorting magnetic activated cell sorting (for example as commercially used in Miltenyi Biotec MACS Technology or Dynal magnetic bead selection), antibody panning and red-cell rosetting. Other methods for isolating cells are contemplated.

T cells expressing a chemokine receptor are as described herein. In certain embodiments, the T cells comprise CD4⁺ cells, CD8⁺ cells, or chimeric antigen receptor T cells.

Chemokine receptors are as described herein. In certain embodiments, an isolated T cell comprises one or more chemokine receptors. In certain embodiments, the isolated T cell comprises a combination of chemokine receptors. Combinations of chemokine receptors are as described herein.

Examples of combinations of chemokine receptors include CCR2 and CXCR3, CCR2 and CCR6, CCR2 and CCR9, CCR2 and CCR10, CCR2 and CXCR4, CCR2 and CXCR6, CCR2 and CXCR5, CCR2 and XCR1, CCR2 and CCR5, CXCR3 and CCR6, CXCR3 and CCR9, CXCR3 and CCR10, CXCR3 and CXCR4, CXCR3 and CXCR6, CXCR3 and CXCR5, CXCR3 and XCR1, CXCR3 and CCR5, CCR6 and CCR9, CCR6 and CCR10, CCR6 and CXCR4, CCR6 and CXCR6, CCR6 and CXCR5, CCR6 and XCR1, CCR6 and CCR5, CCR9 and CCR10, CCR9 and CXCR4, CCR9 and CXCR6, CCR9 and CXCR5, CCR9 and XCR1, CCR9 and CCR5, CCR10 and CXCR4, CCR10 and CXCR6, CCR10 and CXCR5, CCR10 and XCR1, CCR10 and CCR5, CXCR4 and CXCR6, CXCR4 and CXCR5, CXCR4 and XCR1, CXCR4 and CCR5, CXCR6 and CXCR5, CXCR6 and XCR1, CXCR6 and CCR5, CXCR5 and XCR1, CXCR5 and CCR5, and XCR1 and CCR5.

Certain embodiments of the present disclosure provide T cells comprising an exogenous nucleic acid expressing a chemokine receptor and/or comprising an exogenous nucleic acid driving expression of an endogenous chemokine receptor gene.

Cells comprising an exogenous nucleic acid expressing a chemokine (or a combination of receptors) and/or comprising an exogenous nucleic acid driving expression of an endogenous chemokine gene (or a combination of genes) are as described herein. Methods for producing such T cells are as described herein.

Certain embodiments of the present disclosure provide tumour targeting T cells engineered to express a chemokine receptor.

Methods for engineering T cells to express a chemokine receptor(s) are as described herein. Methods for assessing the ability of T cells to a target a tumour are known in the art.

In certain embodiments, the tumour targeting T cells express one or more chemokine receptors as described herein. In certain embodiments, the tumour targeting T cells express a combination of chemokine receptors as described herein.

In certain embodiments, the tumour targeting T cells is a population of T cells, as described herein.

Certain embodiments of the present disclosure use of T cells engineered to express a chemokine receptor(s) for adoptive immunotherapy for preventing and/or treating a cancer.

Methods for using T cells for treating a cancer are as described herein.

Certain embodiments of the present disclosure provide a method of producing therapeutic T cells for adoptive immunotherapy for treating a cancer, the method comprising engineering the cells to express a chemokine receptor.

T cells, and methods for engineering T cells to express a chemokine receptor, are as described herein. Methods for producing T cells are as described herein.

Certain embodiments of the present disclosure provide a chimeric antigen T cell engineered to express a chemokine receptor.

Methods for producing chimeric antigen receptor T cells are as described herein, and are described for example in Pule et al. (2008) Nat. Med 14(11): 1264-1270 herein incorporated by reference.

Certain embodiments of the present disclosure provide products.

In certain embodiments, the present disclosure provides a combination product comprising:
(i) isolated T cells for adoptive immunotherapy to treat a cancer, wherein the T cells express a chemokine receptor; and
(ii) instructions for administering the T cells to a subject.

Isolated T cells expressing a chemokine receptor(s), and methods for using the cells, are as described herein. Methods for isolating T cells are as described herein.

Certain embodiments of the present disclosure provide a method for identifying a chemokine receptor(s) for expression in T cells for treating a cancer. Such methods can be used for example to screen candidate chemokine receptors.

In certain embodiments, the present disclosure provides a method of identifying a chemokine receptor for expression in T cells for adoptive immunotherapy for treating a cancer, the method comprising determining the chemokine expression of the cancer and thereby identifying a receptor to the chemokine for expression in the T cells.

In certain embodiments, the method comprises determining the ability of the chemokine receptor to enhance recruitment/targeting of T cells to a tumour.

Methods for assessing the ability of cells to be recruited to/target a tumour are as described herein.

In certain embodiments, the method comprises use of an animal model. In certain embodiments, the method comprises in vitro studies to assess the efficacy of the chimeric antigen receptor cells.

In certain embodiments, the present disclosure provides kits for performing a method as described herein.

In certain embodiments, the kit comprises one or more reagents and/or instructions for performing the methods, as described herein.

Standard techniques may be used for recombinant DNA technology, oligonucleotide synthesis, antibody production, peptide synthesis, tissue culture, transduction and transfection. Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See for example, Green M R and Sambrook J, Molecular Cloning: A Laboratory Manual (4th edition), Cold Spring Harbor Laboratory Press, 2012, herein incorporated by reference. Methods for using adoptive immunotherapy are known in the art, for example as described in “Adoptive Immunotherapy—Methods and Protocols” (2010) Edited by B. Ludewig and M. W. Hoffman, Humana Press, herein incorporated by reference.

The present disclosure is further described by the following examples. It is to be understood that the following description is for the purpose of describing particular embodiments only and is not intended to be limiting with respect to the above description.

### EXAMPLE 1

### Assessment of Chemokine Expression by Cancers

Chemokines are a family of cytokines that induce chemotaxis of target cells. Currently, there are over 45 known human chemokines and 20 chemokine receptors. Based on the number and spacing of conserved N-terminal cysteine residues that form disulfide bonds, typically chemokines are divided into four groups: (X)C, CC, CXC, and CX3C.

A listing of selected chemokines and their receptors are provided in Table 1.

To determine the profile of chemokines expressed by a primary cancer or a metastatic cancer, initially a biopsy of the cancer may be taken from the patient. The profile of chemokine expression by the cancer can be performed by a variety of methods known in the art, such as by determining transcriptional levels of the chemokines in the cells or determine levels of the chemokines produced by the cells.

An example of a method for determining the chemokine expression is as follows:

(i) Samples

Biopsies from tumours may be obtained by standard techniques and frozen in liquid nitrogen. Sections for in situ hybridisation may be mounted on baked glass slides coated with 3-aminopropyl-triethoxy-silane, air dried, and stored at −70° C. Serial sections may be cut onto poly-L-lysine-coated slides and stored at −70° C.

(ii) RNA Extraction and RT-PCR

Total RNA may be prepared from samples using RNA preparation agents such Tri Reagent (Sigma, Poole, UK). For example, solid tumour biopsies may be homogenised in Tri Reagent using an Ultra-turrax T25 tissue homogeniser (Janke & Kunkel, Staufen, Germany). For RT-PCR and RNase protection analysis, total RNA may be DNase-treated to remove contaminating genomic DNA, using RNase-free DNase I (Pharmacia Biotech, St Albans, UK). cDNA may be synthesised from DNase treated total RNA using the Ready-to-Go™ T-primed First Strand kit (Pharmacia Biotech, UK). Primers for specific chemokines may be designed using sequences submitted to Genbank, using Primer 3.0 software.

A 25 μl volume per sample may used, containing 200 ng cDNA, 1 U AmpliTaq DNA polymerase, GeneAmp PCR buffer, GeneAmp dNTPs (Perkin Elmer, Beaconsfield, UK) and 4 μM each primer. A PCR protocol utilising a GeneAmp® PCR System 9700 thermal cycler with the following: 94° C. (5 min); 35 cycles 94° C. (30 s), 60° C. (30 s), 72° C. (30 s); 72° C. (7 min). PCR products may be electrophoresed through 1.2% agarose gel and visualised by ethidium bromide. Size markers (Gibco BRL, may be used to estimate band sizes. PCR products may be gel extracted and sequenced to confirm their identity.

(iii) RNase Protection Assay (RPA)

Chemokine templates may be obtained from a commercial provider. RPA May be carried out using [α³⁵S]UTP (Amersham International plc, Aylesbury, UK) or [α³²P]UTP. The RNase-protected fragments may be run out on an acrylamide-urea sequencing gel (BioRad Laboratories Ltd, Hemel Hempstead, UK), which is then adsorbed to filter paper and dried under vacuum. Autoradiography may subsequently be carried out.

(iv) In Situ Hybridisation (ISH)

[α35S]UTP-labelled antisense and sense riboprobes may be generated to specific chemokine cDNAs cloned in a suitable (eg pcDNA1 (Stratagene, Cambridge, UK), using Sp6 and T7 RNA polymerases (Promega Ltd, Southampton, UK). Antisense β-actin may be used as a positive control in all experiments. In situ hybridisation may be carried out using the method described in (Naylor et al (1990) Cancer Res. 50: 4436-4440). Image capture may use Image Grabber PCI (Neotech Ltd, London, UK).

(v) Immunohistochemistry

Cryostat sections may be fixed in 4% paraformaldehyde in PBS for 5 minutes. Sections may be pre-incubated with normal rabbit serum (DAKO, Ely, UK) at a 1/25 dilution, for 15 minutes before application of the primary antibody. Sections may then be incubated for 30 minutes at room temperature with a secondary antibody diluted 1/100; then biotinylated rabbit anti-mouse IgG and avidin-biotin-peroxidase complex (both DAKO). The final incubation may be with the chromogen 3,3′-diaminobenzidine tetrahyrdochloride, and. toluidine blue may be used as the counterstain.

(vi) Northern Analysis

Total RNA (15 μg) may be run on a 1% agarose-formaldehyde gel as described in (Turner et al, (1999) Eur J Immunol 29: 2280-2287. Densitometric analysis may be carried out using appropriate software, such as NIH Image 1.61.

In some cases, specific types of cancers will be known to be associated with the expression of certain chemokines, and accordingly there will not be a need to determine the type of chemokine receptor expressed by the T cells to be used for treatment.

Alternatively, transcriptome profiling can be used to profile cancers for their expression of chemokines. Transcriptome profiling is as described, for example, in Wang et al. (2009) Nature Reviews Genetics 10: 57-63.

### EXAMPLE 2

### Cloning and Expression of Chemokine Receptors

(i) Cloning of Chemokine Receptors

Methods for cloning and performing recombinant DNA technology are generally as described in Green M R and Sambrook J, Molecular Cloning: A Laboratory Manual (4th edition), Cold Spring Harbor Laboratory Press, 2012., herein incorporated by reference, and Ausubel et al., Current Protocols in Molecular Biology (2011), John Wiley & Sons, Inc., both of which are herein incorporated by reference.

Chemokine receptors for cloning may be obtained either by producing a cDNA copy from a suitable cell type expressing an appropriate mRNA or from a commercial source, such as Origene (for example CCR2 (NM_000647) human cDNA ORF clone Cat. #RG221234; CXCR3 (NM_001504) human ORF clone Cat. #RC206596; CCR6 (NM_004367) human ORF clone Cat #RC217036; CCR9 (NM_006641) human ORF clone Cat. #RC210246; CCR10 (NM_016602) human ORF clone Cat. #RC222924; CCR5 (NM_000579) human ORF clone Cat. #RC223291; CXCR4 (NM_003467) human ORF clone RC202069; CXCR6 (NM_006564) human ORF clone Cat. #RG206517).

For example, the accession number for human CCR2 is HGNC:1603. The sequence for isoform A for the mRNA is NM_01123041.2 and the protein is NP_001116513.2. The sequence for isoform B for the mRNA is NM_011233396.1 and the protein is NP_001116868. The CCR2 gene, mRNAs and encoded proteins in other species may be identified by a method known in the art, such as the BLAST suite of algorithms, for example as described in Altschul, S et al. (1990). “Basic local alignment search tool”. Journal of Molecular Biology 215 (3): 403-410. This information can be used to produce a cDNA clone for use in expressing a chemokine receptor in T cells.

The coding regions for the chemokine receptors may be cloned into a suitable vector for expression in T cells. Vectors for expressing proteins/polypeptides are known in the art, and include viral expression vectors and non-viral expression vectors, for example as described in S. C. Makrides (Ed.) “Gene Transfer and Expression in Mammalian Cells” 2003 Elsevier Science B.V.

(ii) Expression in T Cells

Methods for expressing surface receptor genes in T cells are known in the art, for example as described in Bilal et al. (2015) Immunology and Cell Biology 93: 896-908.

Vectors for expressing proteins/polypeptides are known in the art, and include viral expression vectors and non-viral expression vectors, for example as described in S. C. Makrides (Ed.) “Gene Transfer and Expression in Mammalian Cells” 2003 Elsevier Science B.V. Vectors may utilise expression of a fluorescent protein to identify transfected cells.

(ii) Purification and Growth of Human CD4⁺ Peripheral Blood T Cells

Methods for purifying CD4⁺ or CD8⁺ cells are known in the art. Various commercial kits (eg from Miltenyi Biotec, San Diego, Calif., USA) are available for purifying these cells from suitable sources. For example, a kit for isolating CD8⁺ cells may be obtained from Miltenyi Biotec (CD8⁺ T Cell Isolation Kit, human Cat #130-096-495) and an a kit for isolating CD4⁺ cells may be obtained from Miltenyi Biotec (CD4⁺ T Cell Isolation Kit, human Cat #130-096-533).

For example, peripheral blood mononuclear cells may be obtained from whole blood of healthy donors using leukocyte reduction system cones. Negative selection of primary human CD8⁺ T cells or CD4⁺ T cells may be performed using a CD8⁺ T cell Isolation Kit or a CD4⁺ T cell Isolation Kit II (Miltenyi Biotec, San Diego, Calif., USA) to achieve >98%purity.

Isolated CD8⁺ or CD4⁺ T cells may be subsequently activated with magnetic Dyna beads (Invitrogen, Carlsbad, Calif., USA) crosslinked with anti-CD3 (OKT3, BioLegend, San Diego, Calif., USA) and anti-CD28 (CD28.2, BioLegend) in the presence of 100 U ml⁻¹ recombinant IL-2. Cells were cultured at 37° C. and 5% CO₂ in complete RPMI 1640 (RPMI medium supplemented with 10% fetal bovine serum, 50 U ml⁻¹ penicillin, 50 μg ml⁻¹ streptomycin, and 2 mm l-glutamine) (Gibco, Carlsbad, Calif., USA). For flow cytometry analysis or cell sorting, cells may be removed from magnetic beads and immediately analyzed or sorted.

Flow Cytometry

Cells (2×10⁶) may be washed in fluorescence-assisted cell sorting buffer (phosphate-buffered saline, 10% fetal bovine serum and 0.05% sodium azide), and then resuspended in fluorescence-assisted cell sorting buffer to a concentration of 1×10⁶ cells per ml to probe for GFP/YFP expression. For surface staining, primary CD4⁺ T cells may be washed in fluorescence-assisted cell sorting buffer, and then stained with anti-CD44 or anti-CD69 conjugated to Alexa-fluor 647 (Biolegend), CD45RO conjugated to PE-Cy5 (BD Pharmingen, San Diego, Calif., USA). For CD4 staining, cells may be first stained with primary anti-CD4 (clone RPA-T4, Biolegend) and then with secondary Alexa 488 (Biolegend). Cells may be left on ice for 30 min during staining while gently vortexing every 10 min. Cells may be washed, and the MFI of each sample obtained using a suitable flow cytometer (BD Biosciences, San Jose, Calif., USA) and software.

Similar protocols may be used for CD8⁺ cells.

Viral Production and Purification

Eighteen to 24 h prior to transfections, 3 7.5×10⁶ 293T cells may be seeded in culture dishes at 37° C. and 5% CO₂ in complete Dulbecco's modified Eagle's medium (Dulbecco's modified Eagle's medium supplemented with 10% fetal bovine serum, 50 U ml⁻¹ penicillin, 50 μg ml⁻¹ streptomycin, 1× MEM NEAA and 2 mM l-glutamine) (Gibco). On the day of transfection, the medium may be replaced with fresh complete Dulbecco's modified Eagle's medium.

To generate lentiviral particles, 293T cells may be transfected utilizing calcium phosphate method. In 10-cm plates may be mixed in 0.5 ml of 1× HBS buffer (HEPES free acid 21 mm, NaCl 137 mM, D+Glucose monohydrate 5 mM, KCl 50 mM, Na₂HPO₄ 0.35 mM, pH 7.5) and 30 μl of sterile 2.5 M CaCl₂ added drop-wise to the HBS-DNA solution, mixed by pipetting and then incubated for 20-30 min at room temperature. The HBS-DNA-CaCl₂ mix may then be added drop-wise around the 293T plate and incubated for 15-20 h.

Next, transfection medium may be replaced with fresh medium. Because some viral production initiates shortly after transfection, transfection medium may be replaced at earlier time points to prevent viral loss. Viral containing supernatant may then be subsequently harvested every 24 h for 2 days, and filtered through 0.45 μm Durapore Millex (Millipore) filters. Filtered supernatant may then be divided into round-bottom polycarbonate high-speed tubes (Nalgene-Oak ridge, Thermo Scientific, Waltham, Mass., USA). The tubes may be centrifuged at 4° C. for 1.5-2 h at 48 000 g in Sorvall RC6-Plus centrifuge (Thermo Scientific) (SS-34 Rotor). The tubes are then handled carefully as to not resuspend the viral pellet, and the supernatant aspirated using sterile glass Pasteur pipettes. The pellets may be resuspended in 0.5-2 ml complete RPMI.

Transduction of Primary Human CD4⁺ T Cells

For primary human CD4⁺ T-cell transductions, purified cells may be activated for 1 day with magnetic Dyna beads containing anti-CD3 and anti-CD28 in the presence of IL-2. Next, 10-25×10⁶ cells may be incubated in 1-1.5 ml of concentrated viral supernatant in 25 cm² flasks in the presence of 8 μg ml⁻¹ Hexadimethrine bromide (Sigma Aldrich) with periodical mixing. IL-2 plus beads (100 U ml⁻¹) containing anti-CD3 and anti-CD28 are present during the transduction to preserve the activation status of the cells. Two to 5 ml of complete RPMI medium, Hexadimethrine bromide and IL-2 may be added to the cells every 24 h or after medium becomes visibly yellow. At this point, the flasks may be placed horizontally to reduce cell packing and allow better expansion of the cells. After 72-96 h, cells may be resuspended in fresh complete RPMI without the beads or IL-2 for analysis.

Electroporation of Primary Human CD4⁺ T Cells

Primary human CD4⁺ T cells may be activated for 1-4 days and then magnetic beads removed prior to transfections. CD4⁺ T cells (5×10⁶) may be placed in 100 μl electroporation buffer. Next, 5-20 μg of a plasmid carrying GFP may be added to the cell reagent solution. If using a plasmid that does not carry a fluorescent marker, co-transfection with a GFP carrying plasmid can be used to determine general transfection efficiency. Immediately, the cell-reagent mix may then be aliquoted into a 0.2 cm Lonza or Bio-Rad Gene Pulser cuvette. The cells are then electroporated using Lonza's T-cell programs (such as T-23 or U-14). Cells are immediately removed from the cuvette and then cultured in 5 ml complete RPMI medium in the presence of 100 U ml⁻¹ IL-2. GFP fluorescence may be analyzed after 24-48 h post transfection.

Similar protocols may be used for CD8⁺ cells.

Enrichment of Cells

Flow cytometry may be used to enrich for T cells expressing a specific chemokine receptor, using for example, antibodies to the receptors. Antibodies to chemokine receptors are known in the art and commercially available, for example antibodies for use in flow cytometry against chemokine receptor (eg as available from R&D Systems; CCR2 Cat. #FAB151A, FAB151C, FAB151G, FAB151N; CXCR3 Cat. #FAB160A, FAB160C, FAB160F, FAB160N, FAB160P; CCR6 Cat. #FAB195A, FAB195C, FAB195F, FAB195P; CCR9 Cat. #FAB179A, FAB179A, FAB179F, FAB179P, FAB1791A, FAB1791P; CCR10 Cat. #FAB3478A, FAB3478C, FAB3478P; CCR5 Cat. #FAB182F, FAB182P; CXCR4 Cat. #FAB173P, FAB170F, CXCR6 Cat. #FAB699P, FAB699A).

Other markers that may be used to enrich for a specific type of T cells, and are known in the art.

T cells expressing the selected chemokine receptor may be expanded by a method known in the art for use in clinical applications.

Methods for genetic modification of T cells are as described, for example, in Bilal et al. (2015) Immunology and Cell Biology 93: 896-908.

### EXAMPLE 3

### Treating Solid Tumours Using T Cells Expressing a Chemokine Receptor

Regimens for administering T cells will vary depending on the cancer being treated. Methods for clinically administering T cells are known in the art.

For example, selected patients suffering from a cancer expressing a chemokine may be subjected to a single round or multiple rounds of treatment, depending on the efficacy and side effects experienced as a result of the administration of the treatment. In the event of multiple rounds of treatment there may be a predetermined period of time (approximately between 2 and 6 weeks) between each round of treatment permitting assessment of the efficacy of the treatment, and allowing the patient to recover from any adverse effects.

For an adult subject (approximately 60 to 100 kg) each round of treatment may, for example, consist of the administration of up to 6 doses of modified cells interspaced by a 12 hour rest period, with the first round of treatment starting at between 3×10⁹ to 6×10⁹ cells. If no significant adverse events are experienced, the next round may consist of approximately 1×10¹⁰ to 2×10¹⁰ cells with the following round increasing to between approximately 3×10¹⁰ to 5×10¹⁰ cells. The cells can be administered in any suitable pharmaceutical acceptable carrier. However, in the simplest form the cells are suspended in 100 ml of isotonic saline and administered to the patient intravenously over a period of approximately 20 to 30 mins.

Clinical parameters may be used to determine the efficacy of treatment for the selected cancer.

Some cancers are associated with expression of one or more certain chemokines, and therefore T cells expressing a receptor(s) to these chemokines may be used for treatment (see for example Table 2) using a method as described herein.

TABLE 2

| Ligands | Examples of Type of Cancer | Examples of Metastasis Sites |
|---|---|---|
| CXCL5, CXCL6, | Melanoma, breast cancer, ovarian | |
| CXCL8 | cancer, prostate cancer | |
| CXCL1, CXCL2, | Melanoma, breast cancer, ovarian | Lung |
| CXCL3, CXCL5, | cancer, prostate, renal cell | |
| CXCL6, CXCL7, | carcinoma, pancreatic cancer, | |
| CXCL8 | esophageal cancer | |
| CXCL4L1, CXCL4, | Melanoma, breast cancer, | Lung, lymph nodes |
| CXCL9, CXCL10, | colorectal cancer, osteosarcoma, | |
| CXCL11 | ALL, B-cell CLL | |
| CXCL12 | Melanoma, breast cancer, ovarian | Bone marrow, lymph nodes, |
| | cancer, prostate cancer, glioma, | lund, liver, peritoneum |
| | neuroblastoma, squamous cell | |
| | cancer, head and neck cancer, | |
| | esophageal cancer, stomach | |
| | cancer, bladder cancer, pancreatic | |
| | cancer, colorectal cancer, renal | |
| | cancer, osteosarcoma, NSCLC, | |
| | AML, FCCL, ALL, NHL, multiple | |
| | myeloma | |
| CXCL13 | Squamous cell cancer, B-cell CLL, | Bone, lymph node, peripheral |
| | mantle lymphoma | nerves |
| CCL3, CCL4, CCL5, | Colorectal cancer | Liver |
| CCL6, CCL7, CCL8, | | |
| CCL13, CCL14, CCL15, | | |
| CCL16, CCL23 | | |
| CCL2, CCL5, CCL7, | Breast cancer, prostate cancer, | Bone marrow, lung |
| CCL8, CCL13, CCL16 | multiple myeloma | |
| CCL4, CCL5, CCL7, | Renal carcinoma, CTCL | |
| CCL11, CCL13, CCL15, | | |
| CCL24, CCL26, CCL28, | | |
| CCL17, CCL22 | CTCL, ATL, ATLL, PTCL | Skin |
| CCL3, CCL4, CCL5, | Breast cancer, colorectal cancer | Liver, lung |
| CCL7, CCL14, CCL16 | | |
| CCL20 | Colorectal cancer | Liver |
| CCL19, CCL21 | Melanoma, breast cancer, non- | Lymph nodes |
| | small cell lung cancer, lung cancer, | |
| | head and neck cancer, esophageal | |
| | squamous cell carcinoma, stomach | |
| | cancer, gastric carcinoma, | |
| | colorectal cancer, B-cell CLL, | |
| | CLL | |
| CCL25 | Melanoma, breast cancer, prostate | Small intestine |
| | cancer, T-ALL | |
| CCL27, CCL28 | Melanoma, CTCL | Skin |
| CX3CL1 | Breast cancer, prostate cancer, | Bone marrow, peripheral |
| | colorectal cancer, PDAC | nerves, brain |
| CCL11, CCL12 | Breast cancer, NSCLC, | Lung, bone marrow, liver, |
| | rhabdomyosarcoma | brain |
| CCL18 | Breast cancer | Lung, liver |

In other circumstances, the profile of chemokine expression for a cancer in a subject may first be determined, and use of T cells expressing a receptor to that chemokine may then be used for treatment.

In this case, non-autologous T cells expressing the chemokine receptor may be used for treatment which have been prepared previously. Alternatively, autologous T cells from the subject may be obtained, transfected with a suitable expression construct, tested and expanded for treatment.

### EXAMPLE 4

### CAR T Cells Expressing a Chemokine Receptor to Treat Solid Cancers

Chimeric antigen receptor T cells directed to a specific tumour antigen may be produced by a method known in the art, for example as described in Pule et al. (2008) Nat. Med 14(11): 1264-1270.

Introduction of a chemokine receptor into these CAR T cells may be accomplished as described herein.

CAR T cells expressing a chemokine receptor may be introduced into a subject and used to treat a solid tumour, for example as described in Brawley et. al (2015) J. Clin. Oncol. 33(15): 1688-1696.

### EXAMPLE 5

### Screening for Chemokine Receptors for Use in T Cells for Treating Cancer

The methods as described herein may also be used as the basis for screening for chemokine receptors that can be expressed in T cells for treating cancers.

For example, the chemokine expression associated with a cancer may be determined as described herein.

Dependent upon the chemokine(s) identified as being expressed in a cancer, a receptor(s) to the ligand(s) may be selected for further investigation. Examples of chemokines and associated receptors are provided in Table 2.

A selected chemokine receptor can be introduced into suitable T cells for use in an animal model, such as a mouse model.

The ability of the T cells to treat a cancer in the animal model, and/or to enhance trafficking/recruitment of T cells to a tumour are indicative that the chemokine receptor be a potential therapeutic receptor for expression in human T cells.

In addition, the methodology allows for investigation of modifications to a chemokine receptor to be tested.

### EXAMPLE 6

### IL-17-Producing γδ T Cells Switch Migratory Patterns Between Resting and Activated States

Abstract

Interleukin 17-producing γδ T (γδ T17) cells have unconventional trafficking characteristics, residing in mucocutaneous tissues but also homing into inflamed tissues via circulation. Despite being fundamental to γδ T17-driven early protective immunity and exacerbation of autoimmunity and cancer, migratory cues controlling γδ T17 cell positioning in barrier tissues and recruitment to inflammatory sites are still unclear. Here we show that γδ T17 cells constitutively express chemokine receptors CCR6 and CCR2. While CCR6 recruits resting γδ T17 cells to the dermis, CCR2 drives rapid γδ T17 cell recruitment to inflamed tissues during autoimmunity, cancer and infection. Downregulation of CCR6 by IRF4 and BATF upon γδ T17 activation is required for optimal recruitment of γδ T17 cells to inflamed tissue by preventing their sequestration into uninflamed dermis. These findings establish a lymphocyte trafficking model whereby a hierarchy of homing signals is prioritized by dynamic receptor expression to drive both tissue surveillance and rapid recruitment of γδ T17 cells to inflammatory lesions.

Materials and Methods

Mice: C57Bl/6 and Ly5.1 mice were purchased from Animal Resource Centre (WA, Australia) or bred at the University of Adelaide animal facility. Il17a^{Cre}×Rosa26^{eYFP}, Ccr6^{-/-}, Ccr2^{-/-}, Ccr2^{-/-}Ccr6^{-/-}, Tcrd^{-/-} and C57Bl/6×Ly5.1 (F₁) mice were bred at the University of Adelaide animal facility. Irf4^{-/-}, Irf8^{-/-}, Batf^{-/-} and Lck^{Cre}Prdm1^{fl/fl} mice were bred at the WEHI animal facility. Mice were age- and gender-matched and used at 6-14 weeks of age. Experiments were conducted with approval of the University of Adelaide Animal Ethics Committee.

Disease models: Mice were immunized for chronic EAE by subcutaneous injection of 100 μg of MOG₃₅₋₅₅ (GL Biochem) in phosphate-buffered saline (PBS) emulsified 1:1 in complete Freund's adjuvant, coupled with i.p. injection of 300 ng Pertussis toxin (Sapphire Bioscience) on days 0 and 2. Mice were analysed at clinical scores of 0.5 (onset) and 2-3 (peak) in wild type (WT) mice, where scoring criteria were: 0.5 tremor, 1 partially limp tail, 2 fully limp tail, 2.25 unable to right, 2.5 sprawled hindlimbs, 2.75 one hindlimb paralysed, 3 both hindlimbs paralysed, 3.5 one forelimb paralysed. B16.F10 melanoma cells (mycoplasma free and verified by short tandem repeat) were cultured in RPMI 1640 containing 10% fetal calf serum (FCS) and 5×10⁴ cells were injected subcutaneously into mice at four sites. S. pneumoniae EF3030 was grown to a Dγδ of 0.18 in nutrient broth with 10% horse serum at 37° C. 5% CO2 and stored at −80° C. Stocks were defrosted and 1×10⁶ colony-forming units were delivered intranasally. Bacterial load was determinedby serial dilution of concentrated NW onto blood agar with 5 μg ml⁻¹ gentamicin (Sigma).

Cell preparation: Single-cell suspensions were prepared from lymphoid organs bypressing through 70 μm filters. Peripheral blood was collected into heparinised Vacutainer tubes (BD). Red blood cells were lysed as required. CNS from PBS-perfused mice was pressed through 70 μm filters and then separated over a 30/70% Percoll (GE) gradient at 500 g. The following digestions were performed at 37° C. with 30 U ml⁻¹ DNase (Sigma). Epidermis and dermis from ears or shaved trunk skin were separated by incubation in 0.375% tryspin for 2 h at 37° C., and then digested separately with 85 mgml⁻¹ Liberase™ (Roche) for 1 h. Tumours and perfused lungs were digested in 2 mgml⁻¹ collagenase (Sigma) for 1 h. NP tissue between the nose tip and eyes was dissected following removal of nasal-associated lymphoid tissue, digested with 2 mgml⁻¹ collagenase for 1.5 h and separated by a 40/80% Percoll gradient at 600 g. Livers were pressed through 70 μm filters and then lymphocytes were isolated by a 37.5% Percoll gradient at 850 g. Flushed and longitudinally opened small intestine free of Peyer's patches was washed in PBS then incubated in 5 mM EDTA for 40 min at 37° C. to remove epithelium, before remaining lamina propria was digested with 0.5 mg ml⁻¹ collagenase for 1.5 h at 37° C. and separated over a 40/80% Percoll gradient at 1,000 g to isolate lamina propria lymphocytes. Peritoneal exudate cells were collected by 3×1 ml PBS washes.

Flow cytometry: Single-cell suspensions were stained in 96-well round- or v-bottom plates (Corning) at 2×10⁶ lymphocytes per well using antibodies and other reagents. For intracellular cytokine staining, cells were first incubated in complete IMDM with 20 pg ml⁻¹ PMA (Life Technologies), 1 nM ionomycin (Life Technologies) and 1/1,500 GolgiStop (BD) for 4 h at 37° C. Cells were washed in PBS and stained with Near Infrared fixable dye diluted 1/1,000 (Life Technologies) for 15 min at room temperature. Cells were then washed with FACS buffer (PBS 1% bovine serum albumin 0.04% azide) and blocked with mouse γ-globulin (mγg) (200 μg ml⁻¹) for 5 min at room temperature. All subsequent steps were incubated at 4° C. For purified antibodies, cells were stained with purified antibody for 20-60 min, washed in FACS buffer, stained with secondary antibody in mγg (200 μg ml⁻¹) and normal mouse serum (NMS) (1%) for 20 min, washed in FACS buffer and blocked with rat γ-globulin for 15 min. Cells were stained with directly conjugated and biotinylated antibodies for 20 min. In the case of biotinylated antibodies, cells were then washed in FACS buffer and stained with streptavidin for 15 min. Cells were then washed in PBS 0.04% azide. For intracellular cytokine staining, cells were incubated in Cytofix/Cytoperm (BD) for 20 min, washed in Permwash (BD) and stained with intracellular directly conjugated antibodies for 20 min. For transcription factor staining, cells were incubated in Foxp3 kit perm buffer (eBioscience) for 30 min to overnight, and then washed in Foxp3 kit permwash (eBioscience). Cells were then stained with directly conjugated a-transcription factor antibodies in NMS (2%) and normal rat serum (2%), and then washed in PBS 0.04% azide. All stains were washed in PBS 0.04% azide, resuspended in PBS 1% paraformaldehyde and stored at 4° C. in the dark.

Specificity of α-CCR2 was confirmed by negative staining on Ccr2^{-/-} γδT17 cells: CCR2 and CCR6 gating was determined by relevant isotype controls. For measurement of fluorescence intensity, relevant isotype control geometric mean fluorescence intensity was subtracted from raw geometric mean fluorescence intensity value. For in vivo proliferation analysis, mice were given 2 mg BrdU i.p. and then drinking water with 0.8 mg ml⁻¹ BrdU 2% glucose. Following restimulation and surface staining, cells were permeabilized, DNase-treated and stained with α-BrdU (BD) according to the manufacturer's instructions. For in vitro proliferation analysis, cells were labelled with Cell Proliferation Dye (eBioscience) according to the manufacturer's instructions. Flow cytometry was acquired on a BD LSR II or FACSAria and analysed with FlowJo (Treestar).

γδT17 cell expansion culture and retroviral transduction: Pooled spleen and LN cells were cultured at 1×10⁶ cells per ml in RPMI 1640 containing 10% FCS, antibiotics, 1× Glutamax (Gibco), 10 mM HEPES (SA Pathology), 1 mM sodium pyruvate, 54 pM β-mercaptoethanol and 1× non-essential amino acids (Gibco) with 5 ng ml⁻¹ recombinant (r)IL-23 (eBioscience), 5 ng ml⁻¹ rIL-1β (Miltenyi Biotec) and 10 μg ml⁻¹ α-IFN-γ (BioXCell) in 96-well round-bottom plates coated with 1 μg ml-1 α-TCR-γδ (clone GL3; Biolegend) for 3 days. Cells were washed and re-seeded on fresh plastic at 1×10⁶ cells per ml for a further 3 days as above without TCR-γδ stimulation. Cells were then washed and re-seeded in 20 ng ml⁻¹ rIL-7 (Peprotech) and 10 μg ml⁻¹ α-IFN-γ for a further 3 days. pMIG, pMIG-Rorc and pMIG-Ccr6 (cloned from mouse Ccr6 cDNA) were transfected into EcoPack 2 293 cells (Clontech; mycoplasma free) with Lipofectamine 2000 (ThermoFisher), and supernatant collected after 48 h. γδT17 cells at days 4 and 5 of culture were centrifuged at 2,500 r.p.m. (30° C. for 1.5 h) in supernatant with 8 μg ml⁻¹ polybrene (Sigma) in flat-bottom 96 well trays before being returned to culture.

Adoptive transfers: For Ccr2^{-/-} and Ccr6^{tg} trafficking experiments, 1-2×10⁶ each of in vitro-expanded F1 (CD45.1⁺ CD45.2⁺) and Ccr2^{-/-} (CD45.2⁺), or transduced control and Ccr6^{tg} γδT17 cells (F₁ or WT), were mixed and transferred i.v. into Ly5.1 (CD45.1⁺) recipient mice d5 post-challenge with B16 melanoma, d8-10 post EAE induction or 24 h post-S. pneumoniae infection. γδT17 cell infiltration of target organs was analysed 24-48 h post-transfer, and CD45 congenic ratios were normalized to input sample. For S. pneumoniae Tcrd^{-/-} reconstitution, in vitro-expanded WT and Ccr2^{-/-} γδT17 cells were further purified by MACS (Miltenyi Biotec) before 3×10⁶ cells were transferred into separate Tcrd^{-/-} hosts 24 h prior to infection. For naïve dermis trafficking experiments, 5-10×10⁷ fresh WT or Ccr6^{-/-} lymphocytes or 3×10⁷ 72 h IL-23/IL-1β stimulated WT lymphocytes were transferred into separate unimmunized Ly5.1 mice and analysed 36 h later. Number of recovered cells was normalized to number of γδT17 cells transferred.

ELISA: Tumour and NP supernatants from digested samples and supernatants from filtered CNS were supplemented with protease inhibitors (Sigma) and stored at −80° C. Mouse CCL2 Duoset ELISA (R&D) was conducted according to the manufacturer's instructions.

qPCR: γδ T cells from Il17aCre×Rose26e^{YFP} mice were enriched by MACS using mouse TCRγδ⁺ isolation kit (Miltenyi Biotec, #130-092-125), and then sorted using a BD FACSAria. Naïve CD4⁺ T cells were sorted from WT splenocytes, and skin stromal populations were sorted from digested epidermal and dermal suspensions from WT mice. RNA was extracted from sorted cells using Qiagen RNeasy Micro kit (#74004). For epidermis and dermis, tissues were snap frozen in liquid nitrogen, crushed with mortar and pestle and RNA purified using Qiagen RNeasy Mini kit (#74104) according to the manufacturer's instructions. cDNA was generated with the Roche Transcriptor First Strand cDNA synthesis kit (#04896866001). qPCR was performed with Roche LightCycler 480 SYBR Green I master mix (#04887352001) using primer sequences on a LightCycler 480 instrument (Roche). Relative gene expression was calculated as 2^{-(CT target-CT reference)} where reference was Rp1p0.

Chemotaxis: Splenocytes were rested in complete RPMI for 3-4 h at 37° C., washed and suspended in chemotaxis buffer (RPMI 0.5% bovine serum albumin 20 mM HEPES). γδT17 cells from culture were washed and suspended in chemotaxis buffer. CCL2 or CCL20 were diluted in chemotaxis buffer and loaded into the lower chambers of 96-well 5 mm pore transwell plates (Corning). 2×10⁶ splenocytes or 2×10⁵ in vitro-expanded γδT17 cells were loaded into the upper chambers and plates were incubated at 37° C. for 3 h. Lower wells were harvested and stained for flow cytometry. CountBrite beads (Invitrogen) were added to samples prior to acquisition to normalize event counts. Chemotaxis index was calculated as number of gated events divided by number in 0 chemokine control.

In vitro stimulation: Splenocytes were cultured in complete IMDM (10% FCS, pen/strep, L-glutamine, β-mercaptoethanol) at 2.5×10⁶ cells per ml with 10 ng ml⁻¹ rIL-23 (eBioscience), 10 ng ml⁻¹ rIL-1β (Miltenyi Biotec), 20 ng ml⁻¹ rIL-7 (Peprotech), 10 ng ml1 rIL-12 (R&D), 10 ng ml⁻¹ rIL-18 (R&D) and/or with pre-coating in 1 μg ml⁻¹ α-TCR-γδ (Biolegend) for up to 72 h at 37° C. For mitomycin C pre-treatment, cells were first incubated with 10 μg ml⁻¹ mitomycin C (Sigma) in complete IMDM at 2×10⁷ cells per ml for 2 h at 37° C. before extensive washing.

ChIP-seq analysis: ChIP-seq data for IRF4 in CD8⁺ T cells, BATF in CD8⁺ T cells and IRF4/BATF in Th17 cells were previously published and were obtained from NCBI database using accession codes GSE49930, GSE54191 and GSE40918, respectively. BAM files were loaded and displayed using the IGB genome browser.

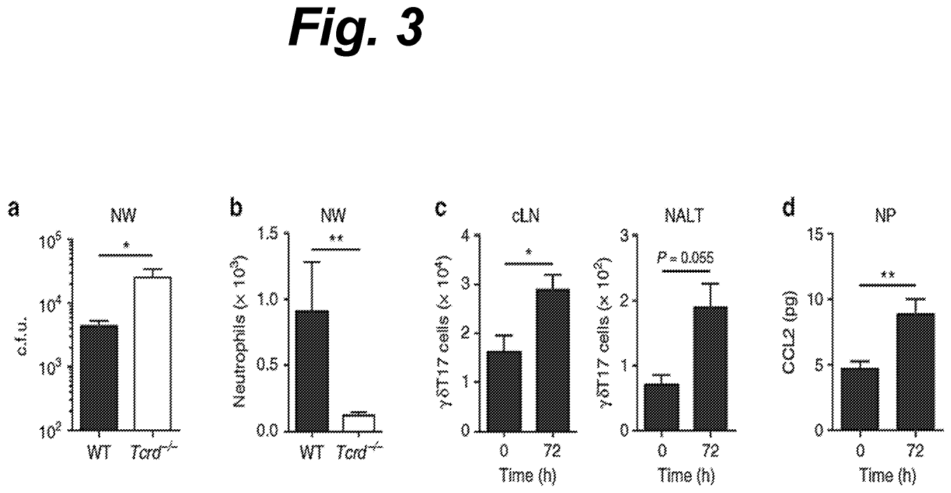

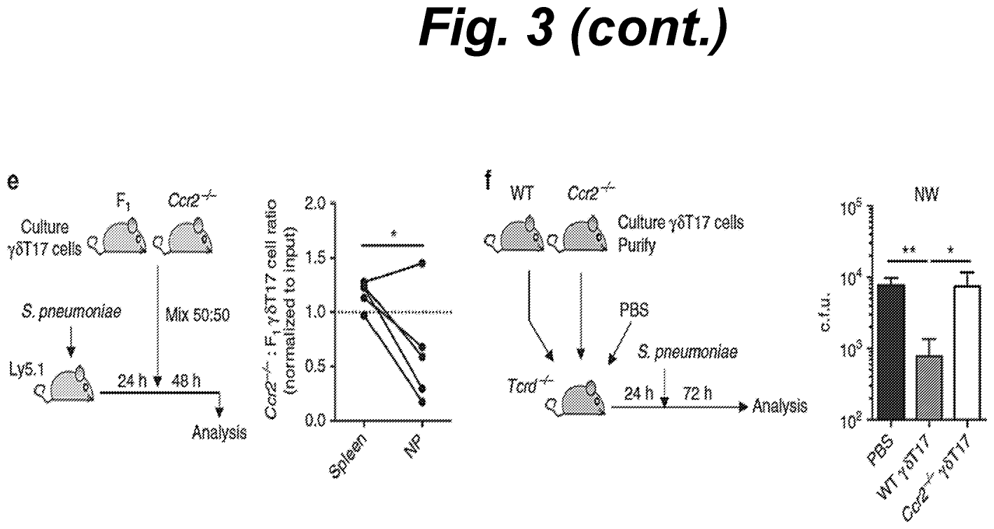

Statistics: Data were analysed with GraphPad Prism 6. Appropriate statistical tests were two-sided and used as indicated in figure legends. *P<0.05, **P<0.01, ***P<0.001, ****P<0.0001. All replicates are biological except in qPCR experiments, where technical replicates are denoted. Sample sizes were determined empirically to ensure adequate power. Tcrd^{-/-} host mice were randomly assigned to groups before receiving adoptive transfers in FIG. 3f. No blinding was utilized. Minimal variance was generally observed between groups; Welch's correction was used in t-tests where standard deviations were significantly different. Most data sets were normally distributed; Mann-Whitney and Kruskal-Wallis tests were utilized where data were not normally distributed.

Results

(i) γδT17 Cells Downregulate CCR6 Upon Activation

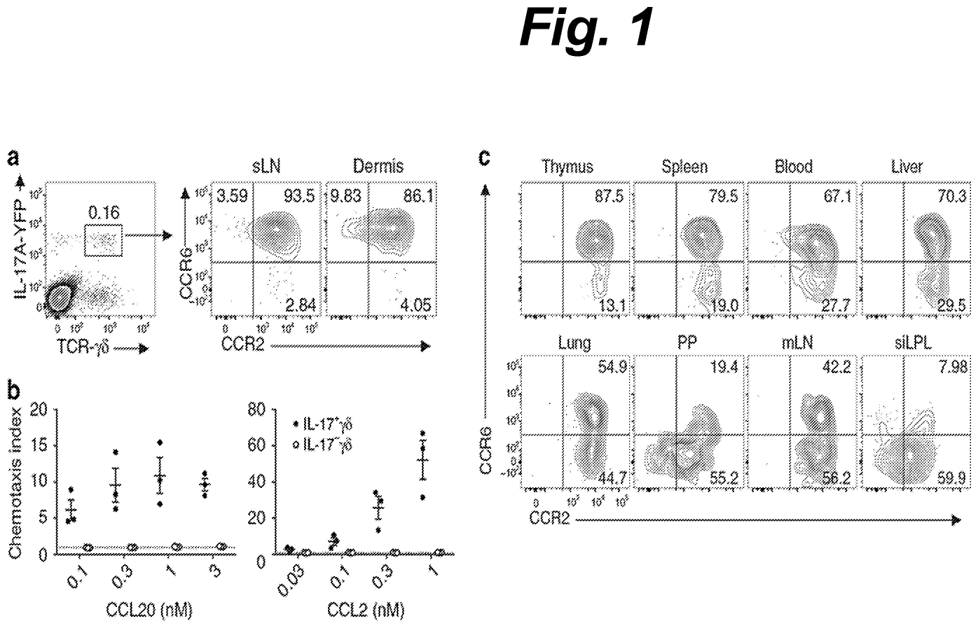

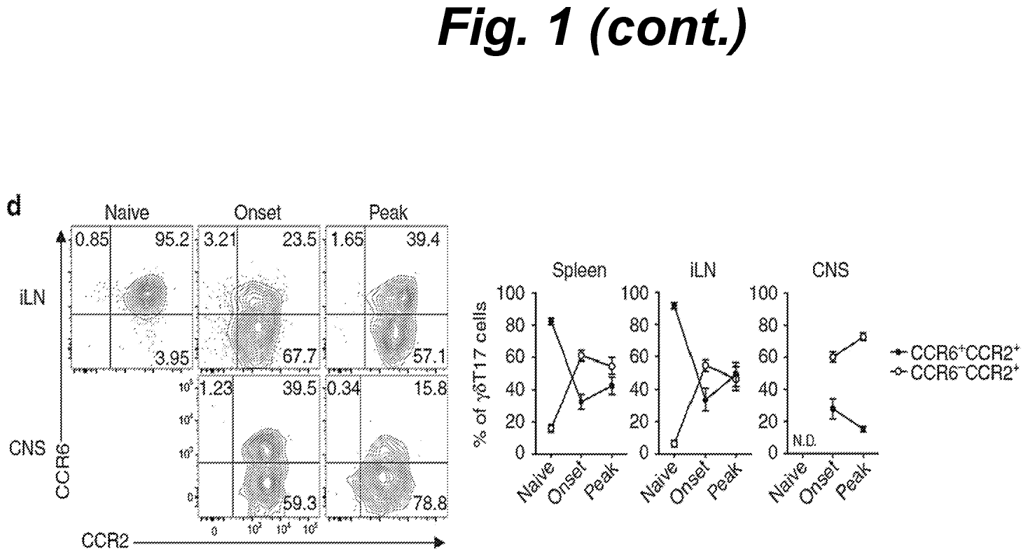

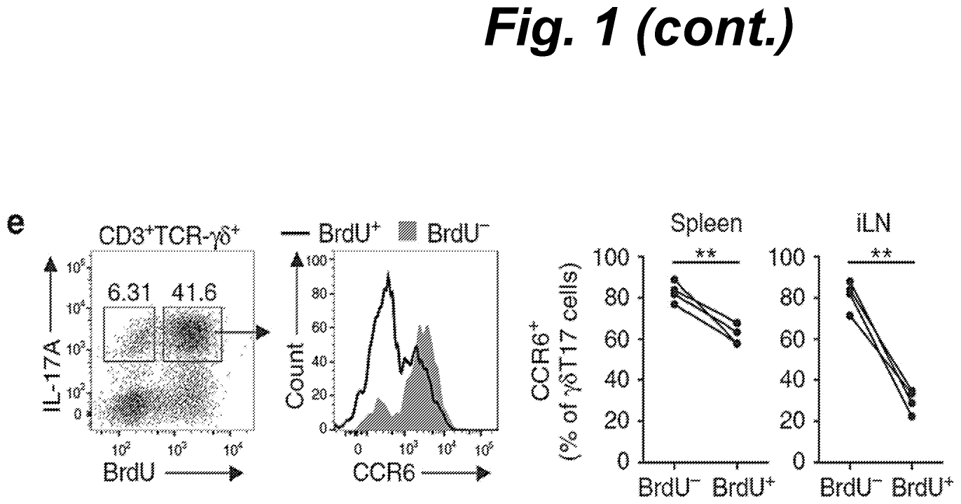

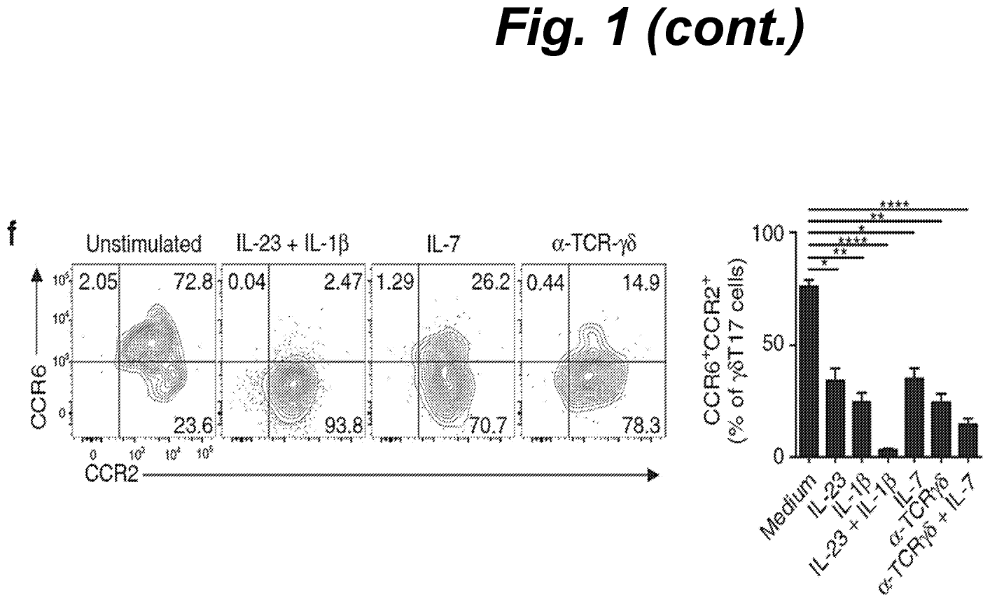

We have found that Th17 cell development during EAE is coupled with a dynamic, temporally regulated switch from CCR6 to CCR2 expression as Th17 cells propagate their differentiation. Expression patterns of CCR6 and CCR2 define distinct effector phenotypes of Th17 cells, with a CCR6CCR2 [symbol] phenotype marking the encephalitogenic granulocyte-macrophage colony-stimulating factor/interferon-γ-producing population. Unlike Th17 cells, γδT17 cell effector function is programmed during thymic development and these cells populate barrier tissues prior to inflammation. Thus, we initially examined CCR6 and CCR2 expression in sLN and dermis in unimmunized Il17a^{Cre}×Rosa26^{eYFP} mice, where Il17a expression drives permanent marking of cells with eYFP. γδT17 cells in these compartments constitutively co-expressed CCR2 and CCR6 (FIG. 1a). Expression of CCR6 and CCR2 was restricted to γδT cells bearing a CD27⁻ CD44^{hi} phenotype, characteristic of γδT17 cells. CCR6/CCR2 co-expression was similar between Vγ4+ and Vγ6⁺ γδT17 cell subsets as distinguished by both Vγ4 expression and CD3/T-cell receptor (TCR) expression level, as previously reported (‘CD3bright staining’) and both receptors were functional as determined by ex vivo chemotaxis (FIG. 1b). However, examination of γδT17 cells from diverse tissues revealed a heterogeneous pattern of CCR6 expression. While thymic and most lymphoid γδT17 cells uniformly expressed both CCR6 and CCR2, populations of γδT17 cells lacking CCR6 expression (CCR6⁻CR2⁺) were prominent in lung and gut-associated tissues (FIG. 1c). As the gut is tonically immunologically active due to interactions with commensal microbiota, we hypothesized that γδT17 cells downregulate CCR6 expression during inflammation.

In support of this idea, activation of sLN and spleen γδT17 cells in vivo during EAE resulted in downregulation of CCR6 expression compared to unimmunized mice (FIG. 1d). CNS-infiltrating γδT17 cells were also largely CCR6⁻. BrdU incorporation revealed that CCR6 expression was downregulated in proliferated γδT17 cells, while BrdU⁻ cells remained CCR6⁺ (FIG. 1e). Other γδ T-cell subsets did not express CCR2 or CCR6 at rest, and did not gain expression of these receptors over the course of EAE. Unlike Th17 cells, γδT17 cells are predominantly activated by TCR-independent signals including IL-23 and IL-1β. In vitro stimulation of lymphocytes with a range of known stimuli including IL-23/IL-1β, IL-23/IL-18, IL-7 and γδ-TCR signalling uniformly repressed CCR6 surface expression in □□T17 cells (FIG. 1f). IL-12 did not impact CCR6 expression, consistent with a reported absence of IL-12R expression by γδT17 cells29. Activation-induced CCR6 downregulation correlated with induction of activation markers CD69 and CD25 and increased CD44 expression, and occurred in both Vγ⁺ and Vγ6⁻ γδT17 cells. In all in vivo and in vitro systems, γδT17 cells maintained high levels of CCR2 following activation, and virtually all γδT17 cells were CCR2⁺ (FIG. 1a,c,d,f). Therefore, γδT17 cells are programmed to co-express CCR6 and CCR2 during development, but lose CCR6 expression upon activation.

(ii) CCR2 Drives cdT17 Cell Recruitment to Inflamed Tissues.

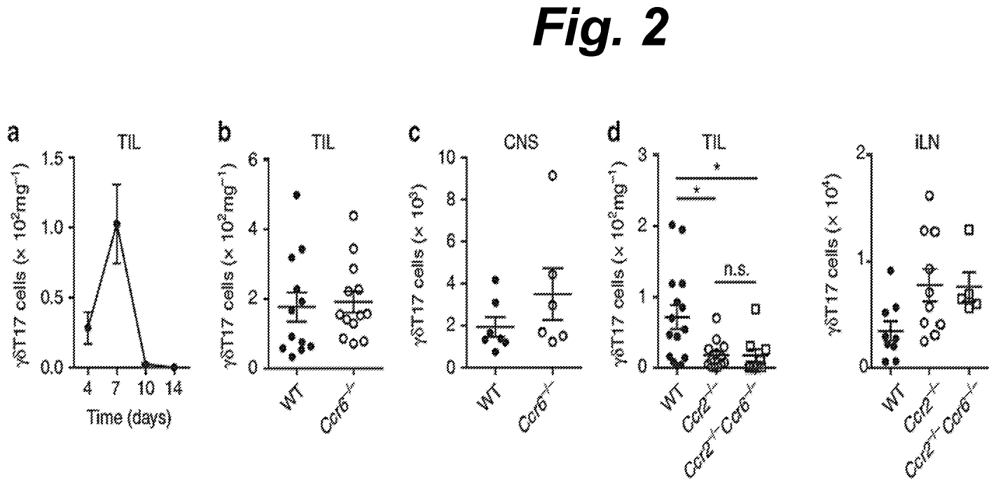

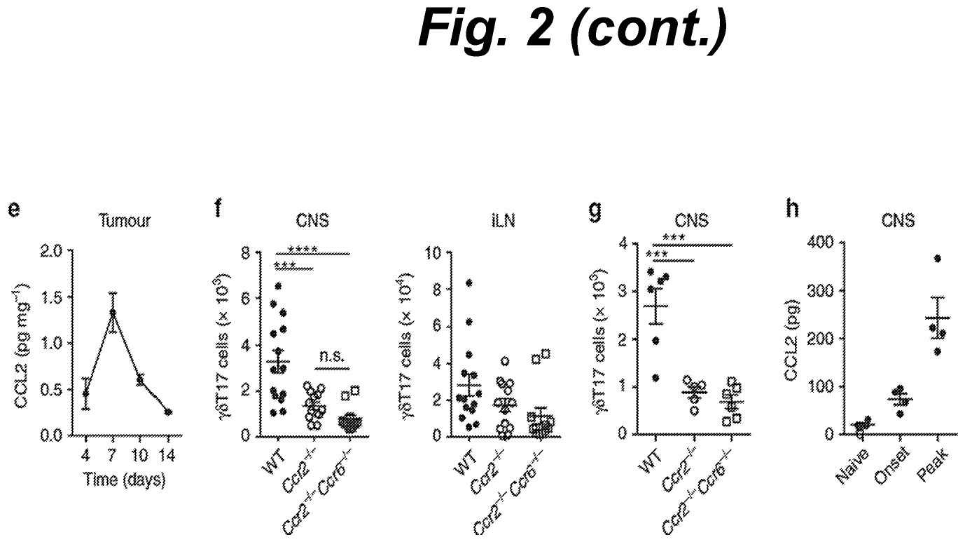

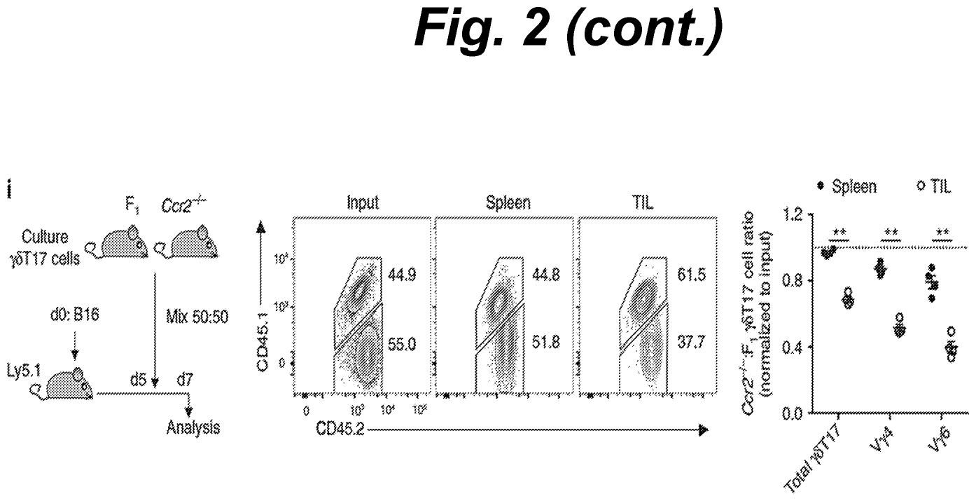

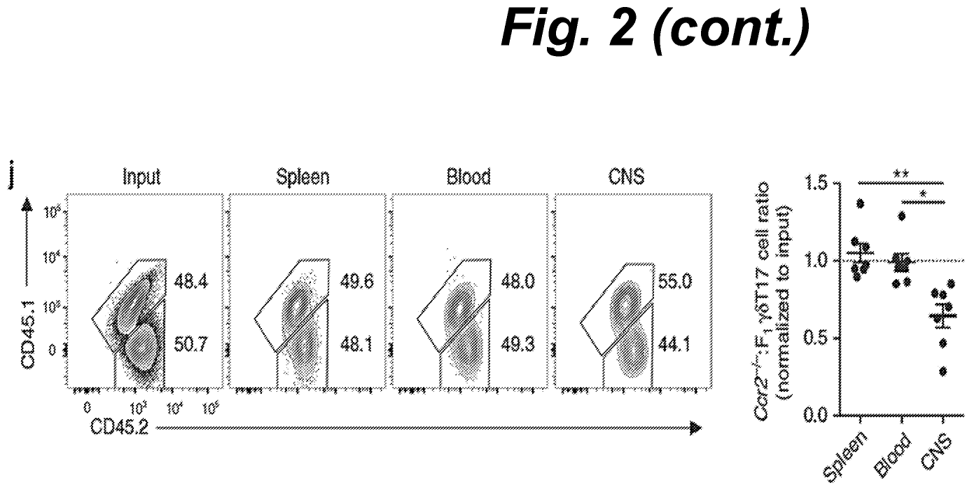

Tissue infiltrating gdT17 cells are best understood in the context of cancer and autoimmunity. gdT17 cells infiltrate B16 melanomas and promote tumour growth30,31, and infiltrate the CNS at disease onset and exacerbate disease pathogenesis during EAE1,32. How gdT17 cells infiltrate these inflammatory lesions is unknown. We thus used these models to investigate CCR6 and CCR2 function in control of gdT17 cell migration during inflammation. Consistent with the observation that activation induces downmodulation of CCR6 expression, Ccr6-deficiency did not affect gdT17 cell infiltration of B16 melanomas (FIG. 2a,b), nor recruitment to the CNS during EAE onset (FIG. 2c). Thus, gdT17 cell trafficking to inflamed tissues in thesesettings occurs independently of CCR6.

In contrast, deficiency of Ccr2 abrogated gdT17 cell infiltration of B16 melanomas but did not affect their expansion in draining lymph nodes (LNs) (FIG. 2d). CCR2-driven infiltration of gdT17 cells was consistent with upregulation of its major ligand CCL2 in tumours (FIG. 2e). Similar results were found in EAE, where Ccr2-deficiency inhibited gdT17 cell recruitment to the CNS at both onset and peak disease (FIG. 2f,g), time points at which CCL2 was induced in the CNS as reported33 (FIG. 2h). CCR2 appeared to operate independently of CCR6 in regulation of gdT17 cell trafficking, as compound deficiency of Ccr6 and Ccr2 (Ccr6/Ccr2/) did not further affect gdT17 cell infiltration in either model (FIG. 2d,f,g). Ccr2/Ccr6/mice exhibited enhanced tumour growth, while Ccr²/and Ccr2/Ccr6/mice had decreased EAE severity. Therefore, to examine the cell-intrinsic requirements of CCR2 for gdT17 cell migration, we developed a novel in vitro expansion protocol to generate large numbers of purified activated gdT17 cells, which contained both Vg4 [symbol] and Vg6 [symbol] subsets and maintained functional CCR2 expression. Equal ratios of in vitroexpanded genetically marked wild type (WT) and Ccr2/gdT17 cells were co-transferred into B16 melanoma-bearing recipients. While donor gdT17 cells recovered from spleen retained the input ratio, Ccr2/gdT17 cells did not migrate efficiently to tumours. This observation was true for both Vg4 [symbol] and Vg6 [symbol] subsets (FIG. 2i). Similar experiments using the EAE model revealed equivalent WT:Ccr2/gdT17 cell ratios in spleen and blood but reduced Ccr2/gdT17 cell recruitment to the CNS (FIG. 2j). Thus, CCR2, but not CCR6, drives activated gdT17 cell migration to inflammatory sites during B16 melanoma and EAE.

(iii) CCR2 is Essential for Protective γδT17 Cell Responses.

The above models involve γδT17 cell infiltration from circulation, as the CNS and tumors lack resident γδT17 cell populations. However, many inflammatory scenarios implicate tissue-resident γδT17 cells, which survey and rapidly defend against infection at barrier surfaces. The extent to which γδT17 cell migration contributes to host defence during ongoing inflammation is unknown. To investigate whether CCR2 also directs tissue-infiltrating γδT17 cells during infection we used experimental Streptococcus pneumoniae infection, immunity to which requires γδT17 cells⁴. Accordingly, Tcrd^{-/-} mice had higher bacterial burden and reduced neutrophils in the nasal wash (NW) than WT at 72 hr post-infection (FIG. 3a-b). S. pneumoniae infection induced γδT17 cell expansion in draining LNs and nasal-associated lymphoid tissue (FIG. 3c). CCL2 was induced in the nasal passages (NP) upon infection (FIG. 3d), and co-transfer of in vitro-expanded WT and Ccr2^{-/-} γδT17 cells into infected mice revealed an intrinsic requirement of CCR2 for γδT17 cell accumulation in NP (FIG. 3e). Thus, CCR2 drives circulating γδT17 cell infiltration of mucosal tissue during S. pneumoniae infection.

To elucidate the ability of recruited γδT17 cells to control infection, we transferred purified in vitro-expanded γδT17 cells into Tcrd^{-/-} hosts prior to S. pneumoniae infection. In this model, tissue-infiltrating γδT17 cells provide the only source of γδ T cell-driven protection. Transfer of WT γδT17 cells reduced nasopharyngeal bacterial burden by tenfold, whereas Ccr2^{-/-} γδT17 cells completely failed to control infection (FIG. 3f). Hence, CCR2 drives recruitment of protective γδT17 cells to the nasal mucosa during S. pneumoniae infection. Collectively, we conclude that γδT17 cell trafficking to diverse inflamed tissues is critically dependent on CCR2 signalling.

(iv) CCR6 Regulates Homeostatic Positioning of γδT17 Cells.

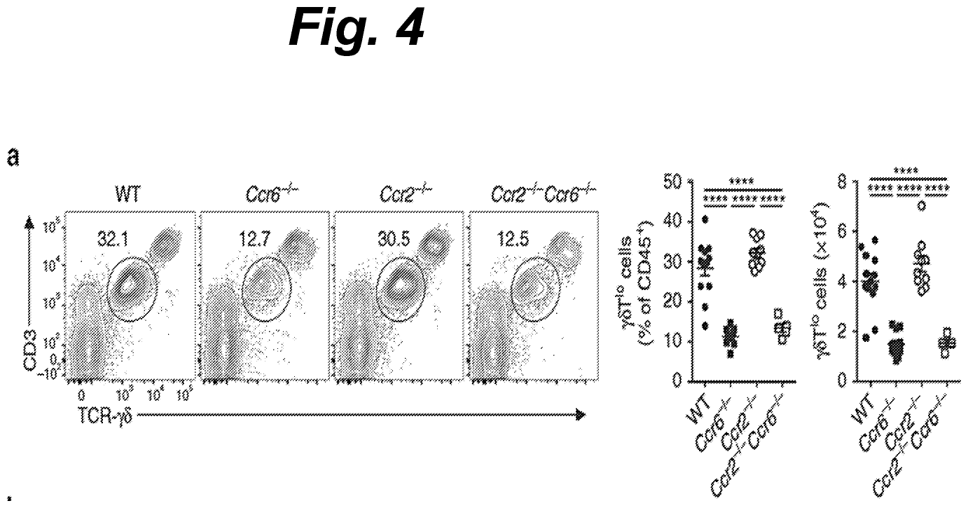

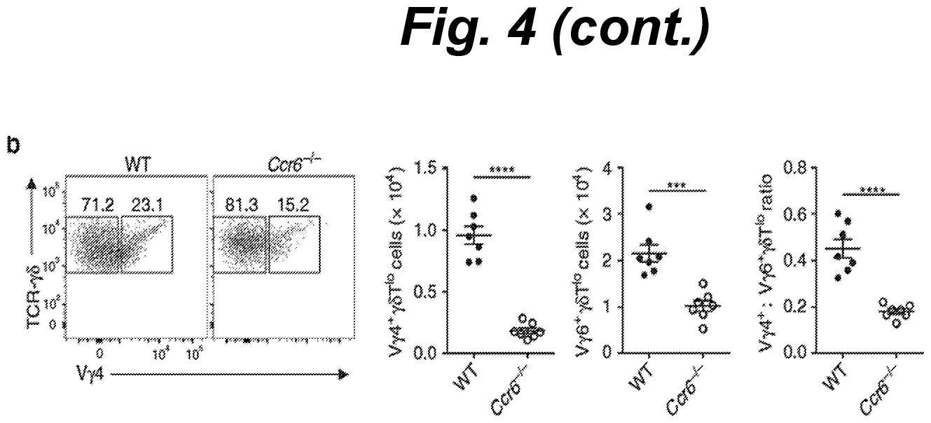

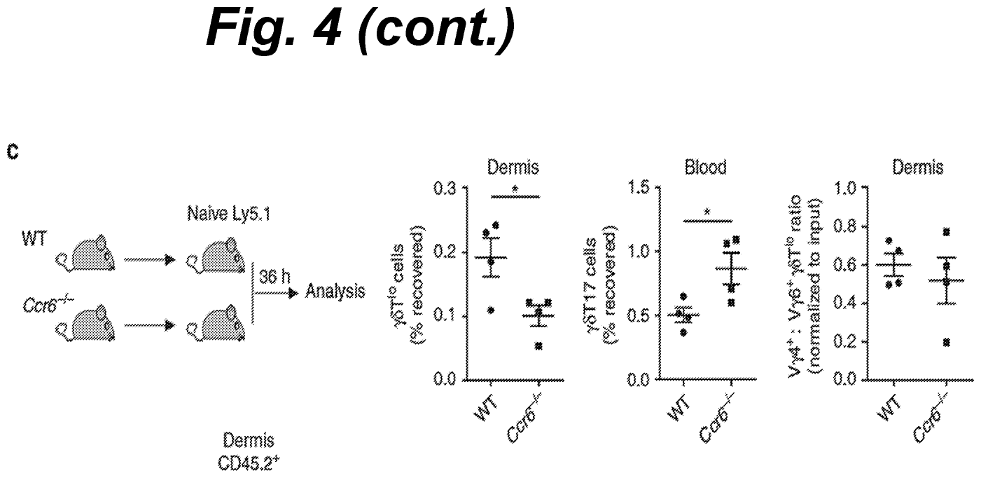

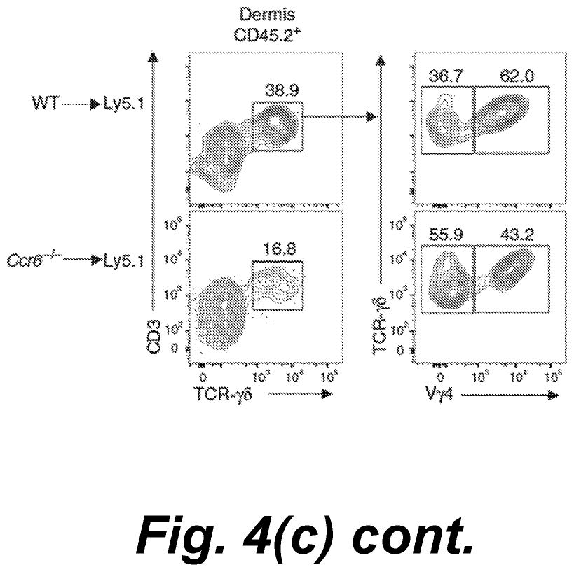

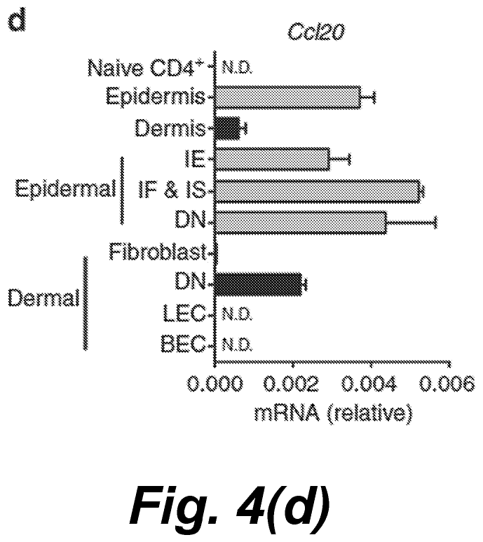

Expression of CCR6 during γδT17 cell thymic development followed by rapid downregulation upon activation suggested that CCR6 plays a more prominent role in regulation of γδT17 cell homeostasis. While CCL20 is induced during inflammation, it is constitutively expressed in barrier tissues including skin, Peyer's patches and large intestine. Both Ccr6^{-/-} and Ccr2^{-/-}Ccr6^{-/-} mice had markedly reduced number and frequency of γδ T cells expressing intermediate amounts of CD3/TCR in the dermis (γδT^{lo}), a population previously reported to produce IL-17 and distinct from TCR^{hi} dendritic epidermal T cells²² (FIG. 4a). We confirmed that γδT^{lo} cells were entirely marked by eYFP in Il17a^{Cre}×Rosa26^{eYFP} mice, despite negligible IL-17A production following ex vivo restimulation. In contrast to a previous report¹⁰, Ccr6-deficiency reduced the number of both Vγ4⁺ and Vγ4⁻ (Vγ6⁻) γδT^{lo} cells, although the ratio was skewed slightly towards Vγ6⁺ cells (FIG. 4b). Examination of other organs revealed that deficiency in Ccr2 had no effect on γδT17 cell homeostasis, while Ccr6-deficiency increased γδT17 cells in the peritoneal cavity. We conclude that CCR6 regulates dermal γδT17 cell residence.

To determine whether CCR6 drove recruitment of circulating γδT17 cells into dermis, we transferred unstimulated WT or Ccr6^{-/-} lymphocytes into naïve mice and tracked their accumulation in dermis. Transferred WT γδT17 cells were substantially enriched in dermis, demonstrating that γδT17 cells can constitutively populate the skin from circulation. In contrast, Ccr6^{-/-} γδT17 cells were defective in infiltration of dermis and pooled in the blood (FIG. 4c). In support of earlier results, both Vγ4⁺ and Vγ4⁻ γδT17 cells were recruited to the dermis, the ratio of which was unaltered by Ccr6-deficiency, suggesting both populations are dependent on CCR6 for circulation-to-dermis trafficking (FIG. 4c). While constitutive expression of CCL20 in epidermis was reported, whether it is expressed in uninflamed dermis is unclear^{34, 38}. We found that Ccl20 was constitutively expressed in the dermis by an uncharacterized CD31⁻CD90⁻ CD140α⁻ stromal population (FIG. 4d). Thus, CCR6 directs homeostatic recruitment of γδT17 cells from circulation into dermis.

(v) IRF4 and BATF Regulate CCR6 Expression in γδT17 Cells

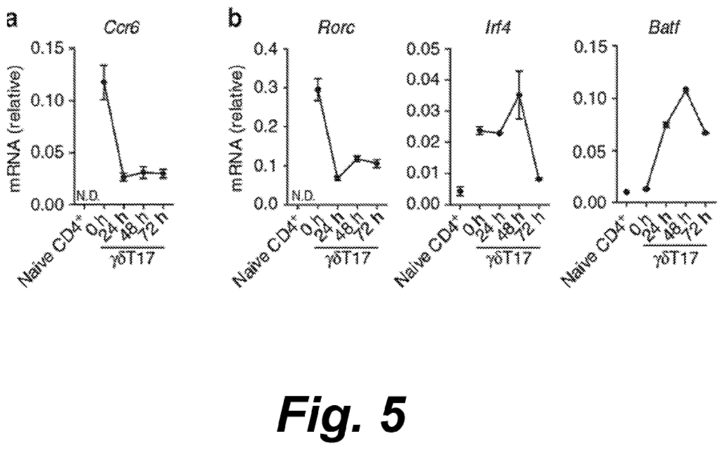

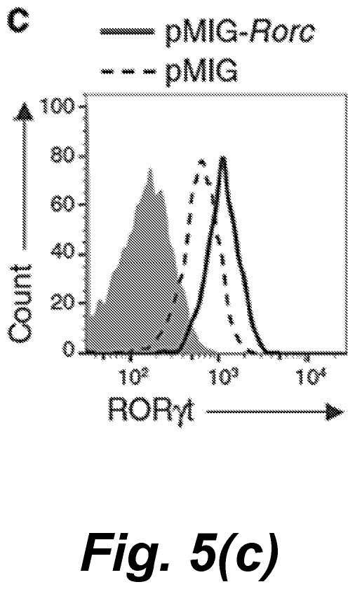

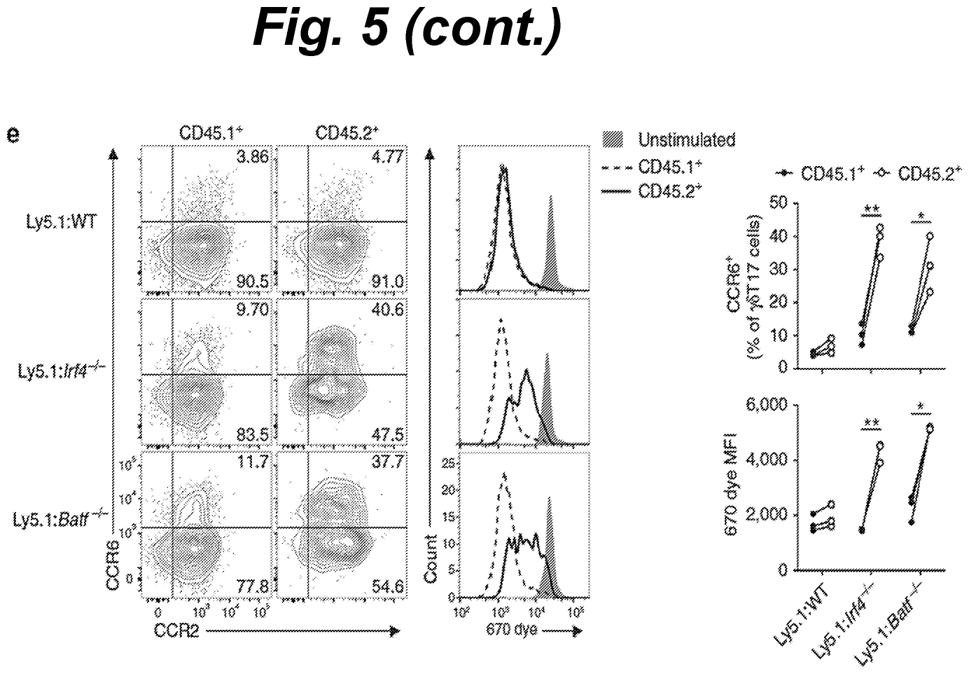

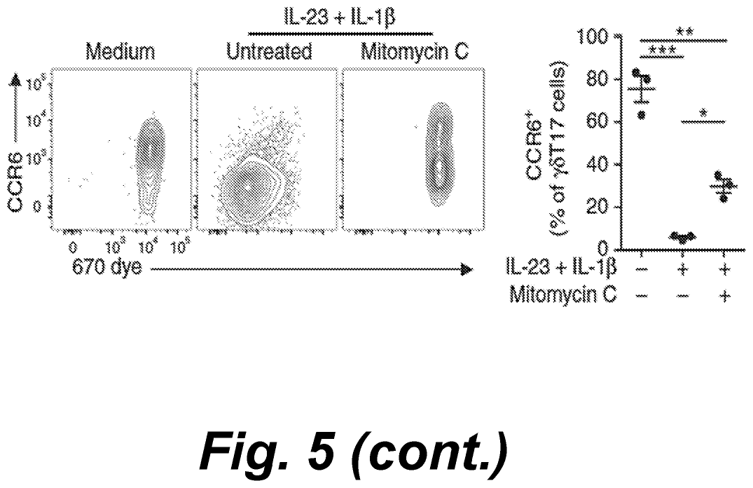

The downregulation of CCR6 upon γδT17 cell activation is surprising, as T cells typically upregulate inflammatory chemokine receptors upon activation. Consequently we investigated the underlying mechanism regulating this process. Ccr6 transcript levels were reduced by fourfold in γδT17 cells within 24 hours of stimulation, whereas Ccr2 expression was maintained (FIG. 5a). This indicated that CCR6 expression was transcriptionally regulated during γδT17 cell activation. We thus examined expression of transcription factors previously implicated directly or indirectly in control of Ccr6 expression, including RORγt, IRF4, IRF8, Blimp1, BATF, T-bet and Eomes. Rorc (RORγt) was highly expressed in resting γδT17 cells but was downregulated by 24 hours of activation. Batf and Prdm1 (Blimp1) were rapidly upregulated by 24 hours, while Irf8 and Irf4 were upregulated by 48 hours, although Irf4 was already present in resting γδT17 cells. Expression of Eomes and Tbx21 (T-bet) at rest or following activation was minimal (FIG. 5b). Therefore, we tested whether RORγt, IRF4, BATF, Blimp1 or IRF8 repressed Ccr6 expression during γδT17 cell activation.

The similar expression kinetics and known Ccr6 regulatory activity of RORγt presented the possibility that its downregulation may result in loss of CCR6 expression. To test this, we retrovirally forced Rorc expression in in vitro-expanded γδT17 cells (FIG. 5c). However, this failed to alter CCR6 expression even in the highest GFP-expressing cells, suggesting that RORγt downregulation is not required for repression of CCR6 in activated γδT17 cells (FIG. 5d).

To determine whether IRF4, BATF, IRF8 or Blimp1 actively repressed Ccr6 expression, we cultured genetically-marked WT and transcription factor-deficient splenocytes with IL-23 and IL-1β. γδT17 cells were present in all strains although at differing frequencies, and homeostatic CCR6 expression was comparable to WT. IRF4- and BATF-deficient γδT17 cells were intrinsically defective in both proliferation and CCR6 downregulation upon stimulation (FIG. 5e). IRF8 and Blimp1 were not required for these processes, although Blimp1 appeared to moderately promote CCR2 expression in activated γδT17 cells. Irf4^{-/-} and Batf^{-/-} γδT17 cells exhibited comparable surface expression of IL-23R and IL-1R1 to WT cells, indicating that maintained CCR6 expression was likely due to defective signalling downstream of IL-23 and IL-1β stimulation. Analysis of our and others' ChIP-Seq datasets in T cells revealed binding of IRF4 and BATF to a shared site in the Ccr6 promoter, suggesting that these factors cooperatively and directly repress Ccr6 in γδT17 cells. To assess whether defective proliferation in absence of IRF4 or BATF was the cause of impaired CCR6 downregulation, dye-labelled WT splenocytes were pre-treated with proliferation inhibitor Mitomycin C prior to stimulation. Although proliferation was effectively blocked, CCR6 downregulation still occurred upon Mitomycin C treatment, suggesting that proliferation and CCR6 downregulation are coincident but independent (FIG. 5f). Together, these data indicate that activation-induced CCR6 downregulation in γδT17 cells is promoted by IRF4 and BATF, and is largely uncoupled from proliferation.

(vi) Loss of CCR6 Promotes γδT17 Cell Homing to Inflamed Tissues

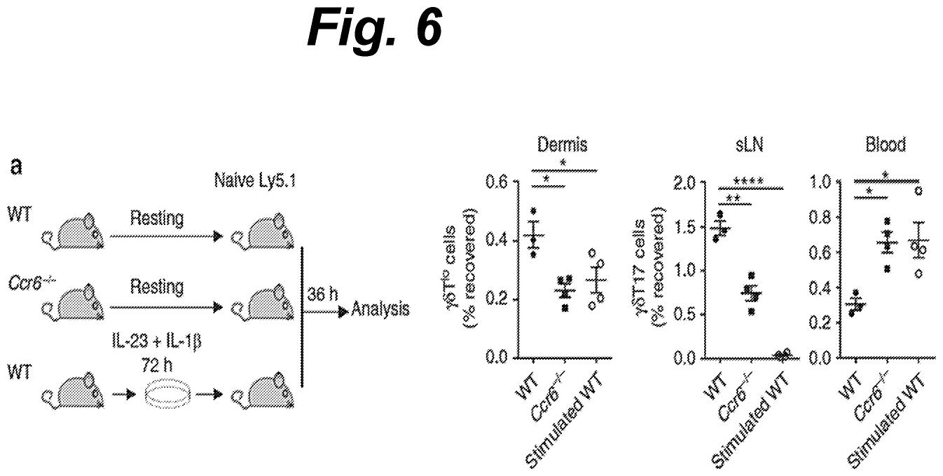

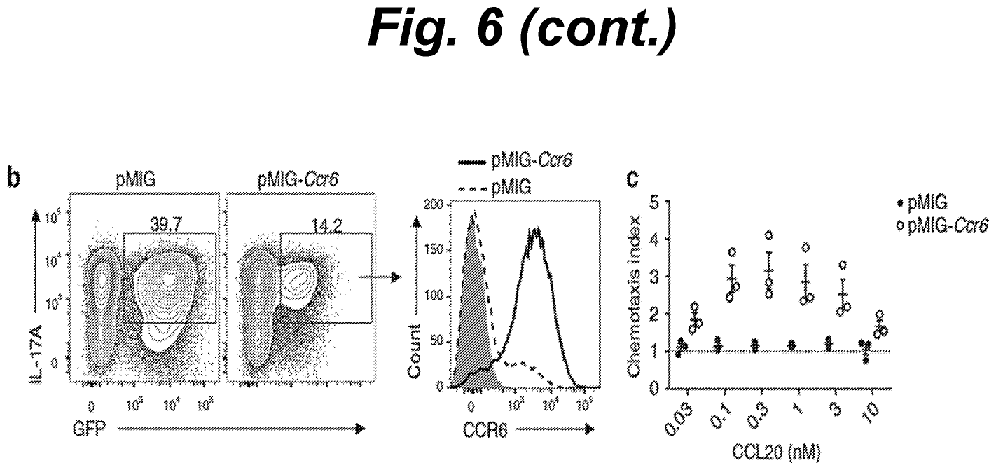

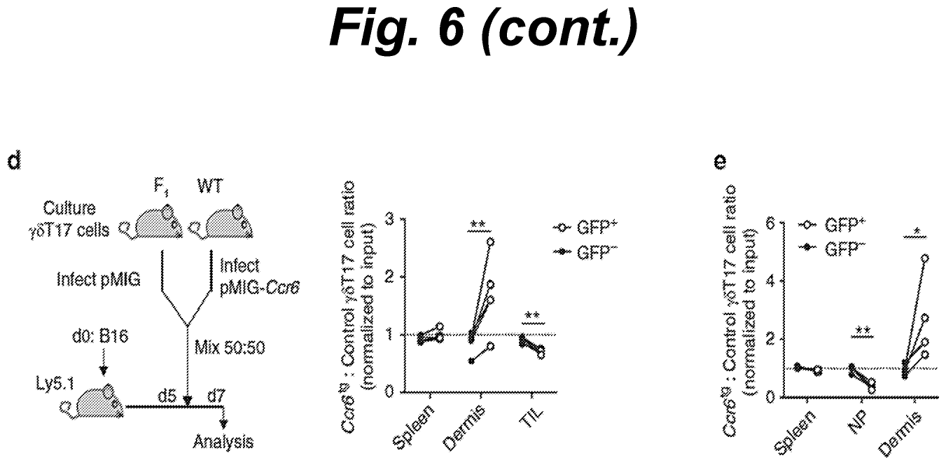

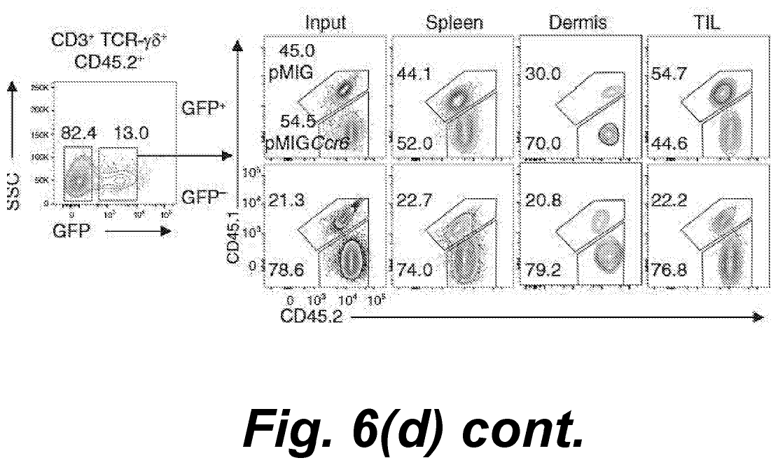

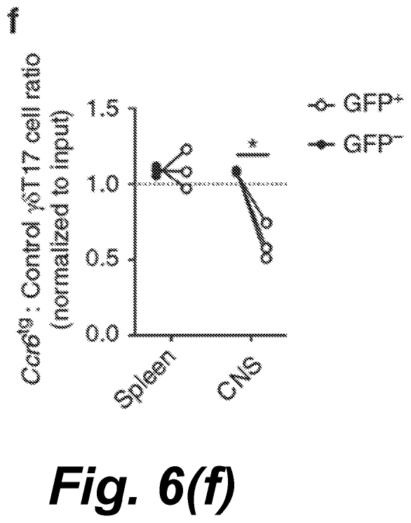

Given the constitutive expression of CCL20 in mucocutaneous sites, we hypothesized that repression of CCR6 during activation enables homing of γδT17 cells toward inflammatory lesions by preventing their accumulation in uninflamed skin. To test this, we first compared the trafficking of in vitro-activated WT γδT17 cells with resting WT and Ccr6^{-/-} γδT17 cells upon transfer into unimmunized hosts. Activated WT γδT17 cells demonstrated the same defect in homing to the dermis as resting Ccr6-γδT17 cells, and both pooled in blood compared to resting WT γδT17 cells (FIG. 6a). γδT17 cells lack CD62L and CCR7 expression, and traffic from skin to LNs in a CCR7-independent manner. Thus γδT17 cell entry to LNs following adoptive transfer likely occurs via afferent lymph draining from dermis. In keeping with this idea, resting Ccr6^{-/-} or in vitro-activated WT γδT17 cells, impaired in their ability to home to uninflamed skin, also accumulated less than resting WT γδT17 cells in sLNs (FIG. 6a). These data are consistent with the notion that activation switches off γδT17 cell homeostatic circulation patterns, enabling directed migration toward inflammatory cues.

To investigate this proposal directly, we studied the migratory patterns of in vitro-activated γδT17 cells retrovirally forced to maintain CCR6 expression. Infection with Ccr6^{tg} virus restored CCR6 expression in activated γδT17 cells, which regained the ability to migrate toward CCL20 (FIG. 6b-c). Genetically marked control- and Ccr6^{tg}-transduced γδT17 cells were mixed 50:50 and transferred into B16 melanoma-bearing recipients. While the input ratio of transferred GFP⁻ cells was maintained in all examined organs as expected, amongst GFP⁺ cells, Ccr6^{tg} γδT17 cells were enriched in the dermis but deficient in tumors (FIG. 6d). Similar results were observed during S. pneumoniae infection: Ccr6^{tg} γδT17 cells were selectively deficient at homing to NP, but accumulated to a greater extent that control-transduced cells in uninflamed dermis (FIG. 6e). Ccr6^{tg} γδT17 cells also homed less efficiently to the CNS during EAE, although subcutaneous CFA immunization precluded analysis of homing to uninflamed skin in this model (FIG. 6f). Together, these experiments demonstrated that activated γδT17 cells with forced CCR6 expression were recruited to uninflamed dermis at the expense of homing to inflamed tissue. Thus, CCR6 downregulation promotes γδT17 cell migration to inflammatory sites.

Discussion

In the present study we define the molecular regulation of γδT17 cell trafficking between resting and activated states. Our data are consistent with a model in which γδT17 cells are imprinted with expression of both CCR6 and CCR2 during thymic development. CCR6 coordinates steady-state recruitment of circulating γδT17 cells into the dermis, where CCL20 is constitutively expressed, thus orchestrating their homeostatic recirculation. Upon activation, γδT17 cells rapidly downregulate CCR6 in an IRF4- and BATF-dependent manner. CCR2 expression is maintained and drives homing of activated γδT17 cells to inflamed tissue during autoimmunity, cancer and infection. CCR6 downregulation is required to promote optimal recruitment of activated γδT17 cells to inflamed tissue, by preventing their diversion and sequestration into uninflamed skin. These data identify a novel mode of lymphocyte trafficking that facilitates both γδT17 cell innate-like surveillance of host tissues and their rapid recruitment to distal sites of ongoing inflammation.

Our data demonstrate that CCR6 coordinates homeostatic recirculation of Vγ4⁺ and Vγ6⁺ γδT17 cells through blood, skin and sLNs. Given that γδT17 cells likely populate sLNs via afferent lymph, our data suggest that LN entry is contingent on first trafficking through the dermis, as Ccr6-deficient γδT17 cells fail to localise to both organs. Thus, in addition to driving infiltration of skin, CCR6 ultimately regulates γδT17 cell homing to sLNs. These findings complement a recent report that CCR6 positions LN Vγ4⁺ γδT17 cells in the subcapsular sinus. Our findings are also in agreement with previous reports of steady-state γδT17 cell circulation, although we show that in addition to Vγ4⁺, Vγ6⁺ γδT17 cells also undergo constitutive trafficking. While most Vγ4⁺ γδT17 cells in LNs do not recirculate within a two week period, approximately 25% undergo extensive circulation¹¹. The non-recirculating population of LN γδT17 cells is likely those positioned in interfollicular zones and SCS, which were proposed to scan for LN-invading microbes. However, the role of the recirculating population of γδT17 cells is unclear. As γδT17 cells do not need to scan LNs for antigen like conventional T cells, why they adopt a constitutive CCR6-dependent circulation loop between tissues and LNs remains to be resolved.

We found that programmed expression of CCR2 equips γδT17 cells with the ability to rapidly home from circulation into diverse inflammatory environments. Our description of CCR2-driven γδT17 cell infiltration of the autoimmune CNS reflects other reports during psoriasis and arthritis. Additionally, CCR2-mediated infiltration of both Vγ4⁺ and Vγ6⁺ γδT17 cells into B16 melanomas suggests that this axis is a universal inflammatory homing signal for γδT17 cells. Our data reveal a redundant role for CCR6 in these processes, due to its immediate downregulation upon γδT17 cell activation. However, these data are inconsistent with reports of CCR6-driven migration of γδT17 cells during skin and liver inflammation. Tissue-specific signals directing maintained CCR6 expression in activated γδT17 cells in these particular scenarios could possibly explain discrepancies between these reports and our data, although this requires further investigation. The function of tissue-infiltrating γδT17 cells during inflammation in barrier tissues, where a resident γδT17 cell population already exists, is unclear. This phenomenon was previously reported during psoriasis, although did not appear to affect disease. Here, we show that γδT17 cells expand in draining LNs and infiltrate nasal mucosa via CCR2 during bacterial infection. In absence of endogenous γδ T cells, transferred circulating γδT17 cells are able to control infection in a CCR2-dependent manner. Thus, we propose that LN-expanded γδT17 cells home to lesions via CCR2 to supplement the local γδT17 cell pool during ongoing tissue inflammation.

Along with our previous work, this study identifies a shared chemokine receptor program used by IL-17-producing cells. We show that like Th17 cells, γδT17 cells express CCR6 early during their effector program, but lose CCR6 expression via IL-23 signalling during ongoing inflammation. Instead, CCR2 is the defining IL-17-program inflammatory homing signal. Whether CCR6 repression in Th17 cells occurs to disrupt barrier tissue-homing and promote recruitment to inflamed tissue, as we show in γδT17 cells, is unclear. Despite γδT17 cell development occurring independently of IRF4 and BATF, we report that these factors suppress Ccr6 expression in γδT17 cells upon IL-23- and IL-1β-driven activation. This is likely to be cooperative, as has been shown during Th17 cell development. Moreover we identified a common binding site for IRF4 and BATF in the Ccr6 promoter, suggesting that repression of Ccr6 expression is directly mediated by these factors. Unlike γδT17 cells however, IRF4 and BATF do not specifically drive the induction of CCR6⁻CCR2⁺ Th17 cells (E. Kara, I. Comerford & S. McColl, unpublished data). Therefore while γδT17 and Th17 cells both lose CCR6 expression during inflammation, the mechanism and function of this process may differ between these populations.

Investigating the trafficking of human γδT17 cells is of clinical relevance as they are increasingly implicated in autoimmunity and cancer, and it will be important to determine whether the model we have established here for murine γδT17 cells applies to humans. Of interest, CCR6 is known to be expressed by both Vδ1⁺ and Vδ2⁺ γδT17 cells in humans, as well as by circulating Vδ2⁺ cells with a skin-homing CLA⁺ phenotype. CCR2 expression has been identified in human γδ T cells, but to our knowledge this has not been examined specifically in IL-17⁺ cells. Whether human γδT17 cells also undergo activation-induced CCR6 downregulation to enhance inflammatory homing has yet to be determined. While the human dermal γδT17 cell compartment is relatively small compared to the murine system, the preponderance of circulating skin-homing Vδ2⁺ cells suggests that similar recirculation mechanisms may also operate in humans. These issues await further experimental resolution.

Our description of γδT17 cell trafficking between steady-state and inflammation presents a novel mode of lymphocyte migration. Conventional T cells downregulate the homeostatic recirculation signal CCR7 and induce expression of inflammatory chemokine receptors upon differentiation into effector subsets, a slow process. In contrast, we show that γδT17 cells constitutively co-express homeostatic and inflammatory homing receptors. The switch in γδT17 cell trafficking from homeostatic to inflammatory programs is solely driven by downregulation of the homeostatic receptor CCR6, rather than induction of additional inflammatory homing receptors. This model likely facilitates immediate homing to inflammatory sites in addition to homeostatic scanning behaviour, consistent with the ‘activated-but-resting’ phenotype of γδT17 cells. Use of CCR6 expression to distinguish resting and activated states may facilitate future investigation of γδT17 cell biology. We conclude that γδT17 cells exhibit a unique bi-phasic trafficking program driven by programmed changes in homing receptor expression to facilitate tissue sentinel responses and rapid homing to distal inflammatory sites.

Although the present disclosure has been described with reference to particular embodiments, it will be appreciated that the disclosure may be embodied in many other forms. It will also be appreciated that the disclosure described herein is susceptible to variations and modifications other than those specifically described. It is to be understood that the disclosure includes all such variations and modifications. The disclosure also includes all of the steps, features, compositions and compounds referred to, or indicated in this specification, individually or collectively, and any and all combinations of any two or more of the steps or features.

Also, it is to be noted that, as used herein, the singular forms “a”, “an” and “the” include plural aspects unless the context already dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Reference to any prior art in this specification is not, and should not be taken as, an acknowledgment or any form of suggestion that this prior art forms part of the common general knowledge in any country.

The subject headings used herein are included only for the ease of reference of the reader and should not be used to limit the subject matter found throughout the disclosure or the claims. The subject headings should not be used in construing the scope of the claims or the claim limitations.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

Future patent applications may be filed on the basis of the present application, for example by claiming priority from the present application, by claiming a divisional status and/or by claiming a continuation status. It is to be understood that the following claims are provided by way of example only, and are not intended to limit the scope of what may be claimed in any such future application. Nor should the claims be considered to limit the understanding of (or exclude other understandings of) the present disclosure. Features may be added to or omitted from the example claims at a later date.

Although the present disclosure has been described with reference to particular examples, it will be appreciated by those skilled in the art that the disclosure may be embodied in many other forms.

1.-25. (canceled)

26. A method of treating a subject suffering from, or susceptible to, a cancer associated with chemokine expressing cells, the method comprising exposing the subject to T cells expressing a receptor to the chemokine and thereby treating the subject.

27. The method according to claim 26, wherein the cancer comprises a cancer having cells expressing a chemokine, a cancer where the tumour stroma expresses a chemokine, or a cancer infiltrated with haemopoietic cells expressing a chemokine.

28. The method according to claim 26, wherein the receptor to the chemokine is one or more of CCR2, CXCR3, CCR6, CCR9, CCR10, CXCR4, CXCR6, CXCR5, XCR1 and CCR5, or a combination of any one or more of the aforementioned chemokine receptors.

29. The method according to claim 26, wherein the T cells comprise CD8⁺ cells, CD4⁺ cells, a chimeric antigen receptor T cells, NKT cells or NK cells.

30. The method according to claim 26, wherein the method comprises administering 10⁹ to 10¹¹ T cells to the subject.

31. The method according to claim 26, wherein the T cells comprise cells expressing an exogenous receptor to the chemokine virally transduced into the cells.

32. The method according to claim 26, wherein the T cells comprise chimeric antigen receptor T cells.

33. The method according to claim 26, wherein the cancer is a solid tumour cancer.

34. The method according to claim 26, wherein the method comprising determining the chemokine expression of the cancer and exposing the subject to T cells expressing a receptor to the chemokine expressed by the cancer.

35. A method of producing therapeutic T cells for adoptive immunotherapy for treating a cancer, the method comprising engineering the cells to express a chemokine receptor.

36. The method according to claim 35, wherein the chemokine receptor comprises one or more of CCR2, CXCR3, CCR6, CCR9, CCR10, CXCR4, CXCR6, CXCR5, XCR1 and CCR5.

37. The method according to claim 35, wherein the T cells comprise chimeric antigen receptor T cells.

38. A T cell produced by the method according to claim 35.

39. A chimeric antigen T cell engineered to express a chemokine receptor.

40. The chimeric antigen T cell according to claim 39, wherein the chemokine receptor comprises one or more of CCR2, CXCR3, CCR6, CCR9, CCR10, CXCR4, CXCR6, CXCR5, XCR1 and CCR5.

41. The chimeric antigen T cell according to claim 39, wherein the cell comprises an exogenous nucleic acid expressing a chemokine receptor and/or an exogenous nucleic acid driving expression of an endogenous chemokine receptor gene.

42. A method of preventing and/or treating a cancer associated with chemokine expressing cells in a subject, the method comprising exposing the subject to chimeric antigen T cells according to claim 39, wherein the cells express a chemokine receptor to the chemokine expressed by the cancer.
